(19) United States
(12) Patent Application Publication
(10) Pub. No.: US 2026/0216324 A1
(43) Pub. Date: Jul. 30, 2026

(54) QS-21 COMPOSITIONS AND PROCESSES FOR PRODUCING THE SAME AND USES THEREFOR

(71) Applicant: Q-VANT BIOSCIENCES, INC., Forestdale, MA (US)

(72) Inventors: Elias ATALA RIFFO; Hans KONSENS CAMPOSANO; Juan Jose ALBARRAN; Cristobal SILVA MIRANDA; Doug KLAIBER, Forestdale, MA (US)

(21) Appl. No.: 19/144,124

(22) PCT Filed: Dec. 22, 2023
(86) PCT No.: PCT/US2023/085646

§ 371 Date: Jun. 27, 2025

Related U.S. Application Data
(60) Provisional application No. 63/435,912, filed on Dec. 29, 2022.

(51) Int. Cl. A61K 39/39; A61K 39/215; A61P 37/04; C07H 15/256

(52) U.S. Cl. CPC A61K 39/39; A61K 39/215; A61P 37/04; C07H 15/256

(57) ABSTRACT

A QS-21 saponin composition used as a standalone immune adjuvant or in an immune adjuvant system or as a cancer therapeutic. The QS-21 saponin composition includes an apiose (V1) isomers and xylose (V2) isomers ratio ranging between 60:40 and 0.1:99.9, wherein both isomers are present. The QS-21 saponin composition having a modified isomeric ratio has been found to provide greater adjuvanticity and no difference in toxicity as compared to a standard QS-21 compound containing apiose (V1) isomers and xylose (V2) isomers in a ratio ranging between 65:35 and 75:25.

### RELATED APPLICATIONS

This application claims priority to, and the benefit of U.S. Provisional Application No. 63/435,912, filed 29 Dec. 2022, the contents of which are incorporated herein by reference in their entirety.

### FIELD OF THE INVENTION

The present invention relates to a QS-21 saponin composition and isomeric mixtures thereof. More particularly, the present invention relates not only to QS-21 compositions and isomeric mixtures thereof, but also to the production of the QS-21 compositions and isomeric mixtures thereof, as well as the use of such QS-21 compositions and isomeric mixtures thereof as a stand-alone immune adjuvant or in an immune adjuvant system for vaccines or as a cancer therapeutic. The QS-21 saponin compositions and isomeric mixtures thereof have been found to provide an unexpected higher immune adjuvanticity as compared to a standard QS-21 saponin composition obtained from the bark of the Quillaja saponaria Molina tree. Furthermore, the process for obtaining either QS-21 saponin or isomeric mixtures of QS-21 saponin from sources other than from the bark of the Quillaja tree is provided. The process allows for the apiose to xylose isomers ratio to fit with the standard QS-21 saponin ratio (65:35) found in the Quillaja tree bark obtained regardless of source of the QS-21 saponin, and also allows for the modification of the apiose to xylose isomers ratio to provide for either apiose-enriched or xylose-enriched QS-21 saponin products and their use as a standalone immune adjuvant or in an immune adjuvant system for vaccines or as a cancer therapeutic.

### BACKGROUND OF THE INVENTION

Saponins have been obtained from the bark of the tree Quillaja saponaria Molina. The term “saponin” as used herein includes glycosidic triterpenoid compounds (also known as triterpene glycoside compounds) which produce foam in aqueous solution, have hemolytic activity in most cases, and possess immune adjuvant activity. The term “saponin” also encompasses biologically active fragments of the above compounds. It will be appreciated that the term “QS” refers to Quillaja saponin.

Quillaja saponins are structurally distinct from the saponins derived from other plant species. Two structural features that distinguish Quillaja saponaria saponins from those of other plant species are a fatty acid domain and a triterpene aldehyde at carbon 4 of the triterpene.

The first commercially available Quillaja saponin adjuvants were crude extracts which, because of their variability, were not desirable for use in veterinary practice or in pharmaceutical compositions for man. Subsequent analysis via high-pressure liquid chromatography showed that Quil A was in fact a heterogeneous mixture of structurally related compounds. (U.S. Pat. No. 5,057,540 and others). However, not all of these saponins were active as adjuvants. The original saponins run via HPLC showed 22 saponin peaks that were separable. The compounds found at each of these peaks have been named QS-1 through QS-22.

The four most predominantly identified and purified Quillaja saponins are QS-7, QS-17, QS-18, and QS-21 (in some literature identified as QA-7, QA-17, QA-18, and QA-21). As noted above, these adjuvant saponins have been identified and purified from an aqueous extract of the bark of the South American tree, Quillaja saponaria Molina, and have generally been purified by HPLC and low-pressure silica chromatography and were found to be adjuvant active, although differing in biological activities such as hemolysis and toxicity in mice. In particular, QS-21 and QS-7 were found to be least toxic in mice. (U.S. Pat. No. 6,524,584 and others).

Due to its potent adjuvant activity and low toxicity, QS-21 (commercially available as the “Stimulon®” adjuvant) has been identified as a useful immunological adjuvant. (U.S. Pat. No. 6,524,584 and others). QS-21 is a complex triterpene glycoside of quillaic acid. QS-21 is glycosylated at triterpene carbon 3, triterpene carbon 28, and carbon 5 of the second fatty acyl unit in a fatty acid domain.

Currently, QS-21 is considered a gold standard of immune adjuvants due to its well-balanced immune response and its capacity to activate the human immune system without substantial toxicity. This property allows QS-21 to be a great component of several vaccine formulations for COVID-19, shingles, malaria, and RSV, and offers the potential for many others. For example, U.S. Pat. No. 5,057,540 describes substantially pure saponins which are useful as immune adjuvants. Disclosed as well are immune response-provoking compositions comprising an antigen in admixture with the substantially pure saponins.

In addition, QS-21 has been incorporated into formulations with a sterol, phospholipid and an antigen, to reduce the toxicity but keeping the adjuvanticity. Therefore, QS-21 is not used as a standalone adjuvant as shown, for example, in GSK's shingles or malaria vaccine or in Novavax's Covid-19 vaccine. Rather, GSK has developed AS01™ adjuvant, which is a liposome-based vaccine adjuvant system containing two immunostimulants: 3-O-desacyl-4′-monophosphoryl lipid A (MPL) and the Quillaja saponin QS-21. Also, Novavax has developed Matrix-M™ adjuvant, which is a saponin-based adjuvant made of nanometer particles, cholesterol, and phospholipid. Moreover, U.S. Pat. No. 10,100,078 describes nanoparticles comprising a sterol and a component selected from Quillaja acid and Quillaja saponin, and the use thereof as an adjuvant, especially in vaccines, as carriers for amphipathic or hydrophobic molecules, and as agents for treatment of cancer.

Unfortunately, there remains problems with QS-21. First, it is very difficult to obtain. As noted in all of the current literature, QS-21 molecule is only produced from the bark of the tree Quillaja saponaria Molina. Currently QS-21 obtention involves the use of Quillaja bark obtained from tree specimens that are over 25 years old as the raw material, with a typical yield no greater than 20 Kg of inner bark per tree. Moreover, when it is extracted, it causes the death of the tree. Therefore, the availability of the Quillaja tree forest is diminishing each year. While replanting does occur, it takes periods of 10 years or more to recover those sections of the forest that have been cut. Moreover, to obtain one kilogram of the pure QS-21 saponin, which is far less than the quantity required by the pharmaceutical industry for one year of production of vaccines, it is necessary to exploit more than 400 acres of native Quillaja forest. This exploitation is pushing the native forest out of existence. This makes the ability to produce the necessary QS-21 compounds unsustainable. Given that the current industry also uses only specific bark batches that fit with some chromatographic requirements, the availability of the raw material is substantially limited, resulting in high production costs, low yields and an uncertain supply for the future. Thus, the need arises to produce QS-21 from sources other than the bark of the Quillaja tree.

In order to overcome the supply problem, the industry has begun developing semi-synthetic saponin adjuvants seeking similar adjuvant activity and toxicity. For example, in the U.S. Pat. Nos. 8,283,456, 8,889,842, 9,718,850 and 10,906,926, semi-synthetic saponin adjuvants have been produced for human vaccines. However, to use these semi-synthetic saponin adjuvants in human vaccines, the regulatory process requires further investigations and validations, since the synthetic QS-21 derivatives are considered a new adjuvant by the main health regulatory agencies, including the US Food and Drug Administration (FDA) and have yet to be validated in humans.

More recent studies of the adjuvant saponin QS-21 has identified it as a molecule comprising a branched trisaccharide, tripterpene, linear tetrasaccharide and an acyl chain, which molecule has been found to be isomeric. That is, purification of QS-21 using hydrophilic interaction chromatography (HILIC) resolved into two peaks, QS-21-V1 and QS-21-V2, which have been shown to be isomeric compounds Thus, it is known that there are two distinct but mixed isomers of QS-21 that generally only differ in the terminal sugar residue within the linear tetrasaccharide segment. These isomers are known as apiose (V1) and xylose (V2). For example, currently, QS-21 obtained from the bark of Quillaja Saponaria generally always comprises at least a 65:35 ratio mixture of the apiose:xylose (V1:V2) isomers. It will be appreciated that, due to the natural variation of the raw material, the isomer ratio of standard QS-21 can range from about 65:35 to 75:25 (V1:V2).

A second problem is that, while hydrophilic interaction chromatography (HILIC) resolved the two peaks for the apiose (V1) isomer and the xylose (V2) isomer at a laboratory scale, it did not enable those skilled in the art to produce the compounds at an industrial scale. Nevertheless, analysis of the immune adjuvant QS-21 clearly indicates that it is a mixture of both isomers, apoise (V1) and xylose (V2). That is, QS-21 obtained from the bark of Quillaja Saponaria has been found to comprise a 65:35 mixture of the apiose:xylose (V1:V2) isomers. As shown in U.S. Pat. No. 5,583,112 (Example 11), Kensil et al. separated the apiose and xylose mixtures themselves and has asserted that the purified apiose isomer and, separately, the purified xylose isomer of QS-21 derived from bark are each separately comparable in both adjuvanticity and toxicity to the standard 65:35 (apiose:xylose ratio) QS-21. Thus, the need arises to find ways to modify and produce the QS-21 compound with superior adjuvanticity and with no difference in toxicity. Additionally, no one has been able to separate the isomers at an industrial scale or with any commercial viability.

### SUMMARY OF THE INVENTION

To overcome the problems raised above, the present patent application describes a process to obtain a QS-21 compound from different sources of Quillaja saponaria and a process to obtain a QS-21 compound having a different apiose to xylose (V1 to V2) isomer ratio than that of standard QS-21. The term “standard QS-21” is defined herein to mean the QS-21 saponin composition found in the bark of the Quillaja tree that includes two saponin isomers, namely, apoise (V1) and xylose (V2), commonly found in an apoise (V1) to xylose (V2) ratio of between 65:35 and 75:25.

With respect to the first problem, it has been found that the standard QS-21 composition found in the bark of the Quillaja tree is substantively the same QS-21 compound found in the wood and leaves (i.e., the biomass). Thus, other parts of the Quillaja tree can now be used in the production of QS-21.

With respect to the second problem, it has been found that xylose-rich QS-21 compositions provide better adjuvant activity as compared to the standard QS-21 compositions having a 65:35 apoise:xylose isomeric ratio. That is, QS-21 mixtures or compounds modified to have a ratio ranging from 60:40 to 0.1:99.9 as an apoise:xylose isomeric ratio show better adjuvanticity and no difference in toxicity as compared to standard QS-21, which contain apiose (V1) isomers and xylose (V2) isomers in a ratio of 65:35. More particularly, the ratio of apiose (V1) isomers to xylose (V2) isomers may range from 60:40 to 0.1:99.9, and even more particularly, from 50:50 to 5:95 and, more specifically, from 40:60 to 20:80. The term “modified QS-21” as used hereinafter is defined to mean any QS-21 saponin composition, or mixture of QS-21 saponin compositions, having both the QS-21 apoise (V1) saponin isomer present and the QS-21 xylose (V2) saponin isomer present in a ratio other than the ratio of standard QS-21, which is 65:35, to possibly 75:25. It will be appreciated that the more important modified QS-21 compositions or mixtures of the present invention are those xylose-rich QS-21 saponin compounds, or mixtures thereof, now defined as having both the QS-21 apoise (V1) saponin isomer present and the QS-21 xylose (V2) saponin isomer present in a ratio of A parts apiose isomer to X parts xylose isomer (i.e., A (of V1):X (of V2), wherein: 0.1<A<65 and 35<X<99.9, wherein A+X=100. In other words, the more important modified xylose-rich QS-21 saponins are those having less than 65% apoise isomer present and more than 35% xylose isomer present, but wherein both the apoise isomer and the xylose isomer are present.

In at least one embodiment, the modified QS-21 compound contains 25% apiose (V1) isomers and 75% xylose (V2) isomers, for a V1:V2 ratio of 25:75. These modified QS-21 compounds may be used as a standalone immune adjuvant or in an immune adjuvant system or as a cancer therapeutic.

Another aspect of the present invention is for a process that obtains QS-21, either as modified QS-21 or as standard QS-21 from the entire biomass (i.e., the bark, the wood and the leaves), thereby decreasing production costs, providing sustainable means, increasing the production yields, and increasing the availability and supply of QS-21, which will enable billions of doses for vaccines. By using other parts of the Quillaja tree, including its wood and leaves, the availability of the raw material is increased, which in turn will trigger an increase in production capacities, reducing costs and making the product sustainable, since the extraction of biomass does not cause the death of the tree or provide stress on the native Chilean forest. Also, the generated adjuvant product (which may or may not include the modified QS-21 compounds) are analogs to the current QS-21. At least the modified QS-21 compounds have been shown to provide the same or diminished toxicity, with more stability, better adjuvant activity, more versatility, and prolonged efficacy as compared to the standard QS-21 compound. Again, it is appreciated that the product obtained by the process can have a different isomers ratio with a unique molecular fingerprint and/or to fit with the ratio of the standard obtained QS-21.

For example, QS-21 obtained from bark of Quillaja saponaria comprises a 65:35 mixture of the apiose:xylose isomers. Accordingly, the standard QS-21 is a mixture of both isomers containing 65% apiose (V1) isomers and 35% xylose (V2) isomers. Despite there being no prior art literature that has every used part of the Quillaja tree other than the bark for QS-21 as an adjuvant, it is reasonable to expect that QS-21 obtained from other parts of the Quillaja tree may have a similar apiose to xylose isomer ratio as the standard 63:35 ratio of standard QS-21, which has always been obtained only from the bark of the Quillaja tree.

Importantly, it has been found that different apiose:xylose isomer ratios of QS-21 affect and impact the results of toxicity and adjuvant activity, with it being discovered that xylose-rich (i.e., 60:40 to 0.1:99.9 [V1:V2 ratios]) QS-21 compounds provide improved adjuvanticity and no difference in toxicity as compared to standard QS-21 saponin containing apiose (V1) isomers and xylose (V2) isomers in a ratio range of 65:35 to 75:25.

Thus, at least one embodiment of the present invention may be found in a QS-21 saponin composition modified by its isomeric ratio of QS-21 apiose saponin isomer and QS-21 xylose saponin isomer within the composition to provide a modified QS-21 saponin composition comprising: a QS-21 apiose (V1) saponin isomer and a QS-21 xylose (V2) saponin isomer in a ratio ranging between 60:40 (V1:V2) and 0.1:99.9 (V1:V2), whereas a standard QS-21 saponin composition comprises a QS-21 apiose (V1) saponin isomer and a QS-21 xylose (V2) saponin isomer in a ratio ranging between 65:35 (V1:V2) and 75:25 (V1:V2).

Another embodiment of the present invention may be found in a QS-21 saponin composition, purified from a crude Quillaja saponaria extract, to provide a pure QS-21 saponin composition comprising a QS-21 apiose saponin isomer and a QS-21 xylose saponin isomer in a ratio of (A parts apiose isomer):(X parts xylose isomer), wherein: 0<A<65 and 35<X<99.9, wherein A+X=100; and wherein the pure QS-21 saponin composition is characterized by one or more predominant peaks comprising at least 90%, preferably at least 90% to 95, and more preferably at least 95%, of the total area of all peaks of a chromatogram, excluding the solvent peak, when analyzed on a hydrophilic interaction liquid chromatography (HILIC) using sulfobetaine 250×4.6 mm, 10 μm, 100 Å under Isocratic conditions of a mixture comprising 84% ACN/16% water (5 mM ammonium acetate) at 1 mL min-1 flow rate and 210 nm of wavelength detection.

Still another embodiment of the present invention may be found in a QS-21 saponin composition, wherein the saponin is taken from the bark and at least one other part of a Quillaja plant.

Yet another embodiment of the present invention may be found in an immunogenic composition comprising an adjuvant composition comprising the QS-21 saponin composition as described herein and above, and an immunogen or antigen, or a polynucleotide encoding the immunogen or antigen.

A further embodiment of the present invention may be found a pharmaceutically acceptable immune adjuvant composition comprising the QS-21 saponin composition as described herein and above. The adjuvanticity of the pharmaceutically acceptable immune adjuvant composition can be measured by an assay selected from the group consisting of an assay comprising induction of IgG antibody production, an assay comprising CD4+ T-cell proliferation, and an assay comprising expression of a CD25 activation marker. The pharmaceutically acceptable immune adjuvant composition has found to have increased adjuvanticity and substantially similar or less toxicity compared to an adjuvant composition comprising a QS-21 apiose saponin isomer and a QS-21 xylose saponin isomer in a ratio of between 65:35 and 75:25.

Yet a further embodiment of the present invention may be found in a cancer therapeutic comprising the QS-21 saponin composition as described herein and above.

Still a further embodiment of the present invention may be found in a process for obtaining the QS-21 saponin composition, the process comprising purifying a Quillaja saponaria extract by a high-performance liquid chromatography (HPLC) separation process comprising; and a reverse phase HPLC separation step. The reverse phase HPLC step may comprise one or more of the following: (1) use of a phenyl-hexyl stationary phase, and (2) use of a sulfobetaine stationary phase. The process may further comprise one or more steps selected from:
collecting raw material from pruning trees of the Genus Quillaja;
milling the raw material;
chipping the raw material;
providing an aqueous mixture of the milled and/or chipped raw material;
filtering the aqueous mixture to obtain an aqueous extract;
concentrating the aqueous extract;
clarifying the aqueous extract by eliminating solids and purify the saponins from the extract;
further filtering the clarified extract to produce a crude extract; and
concentrating the crude extract to obtain a saponin extract.

The step of further filtering may include filtering through a press filter to produce the crude extract and treating the crude extract by nanofiltration techniques to increase the saponin content up to 75 to 99% on a dry basis (ODB), while the step of concentrating may include concentrating the crude extract at vacuum, obtaining the saponin extract in an ultra pure form, and treating the ultra-pure saponin extract with a High-Performance Liquid Chromatography (HPLC) separation with orthogonal two sequential steps to obtaining a QS-21 pure fraction

### BRIEF DESCRIPTION OF THE DRAWINGS

For a more complete understanding of the features and advantages of the present invention, reference is now made to the detailed description of the invention along with the accompanying figures in which:

FIG. 1 depicts a chromatographic profile of bark extract on a C18 column, wherein there is difference in retention time between QS-18 and QS-21 of 3 minutes (using a water pulse).

FIG. 2 depicts a chromatographic profile of Q-Vax S (biomass) on a polar C18 column, wherein there is a difference in retention time between QS-18 and QS-21 of 1.6 minutes.

FIG. 3 depicts a chromatographic profile of Q-Vax-S (biomass) extract on Phenyl-Hexyl column, wherein there is a difference in retention time between QS-18 and QS-21 of 2 minutes.

FIG. 4 depicts a chromatographic profile second step in cellucoat column, wherein the peak at 22 minutes corresponds to QS-21.

FIG. 5 depicts a chromatographic profile second step on diol column, wherein the peak at 24 minutes corresponds to QS-21.

FIG. 6 depicts a chromatographic profile second step on sulfobetaine column, 250×4.6 mm, 10 μm, wherein peaks at 19.6 and 22.4 minutes correspond to isomers of QS-21 (i.e., apiose and xylose).

FIG. 7 depicts an overlapped chromatogram of QS-21 standard and impure QS-21 using a sulfobetaine column. As can be seen, the QS-21 standard has two peaks in a ratio of 65:35 based in their relative AUC that correspond to the QS-21 isomers apiose (rT=19.8 min) and xylose (rT=22.1 min), respectively.

FIG. 8 depicts a QS-21 quality control graph from biomass obtained from the second step, wherein QS-21 represents 97.6% of the area.

FIG. 9 depicts a comparison of saponin profiles of extracts obtained from bark (Q-Vax), biomass (Q-Vax-S) and Quil-A (Croda International, plc product).

FIG. 10 depicts a chromatogram obtained for the Quillaja bark extract, wherein chromatography was performed in the industrial liquid chromatography equipment using a 15 cm diameter DAC column filled with 10 μm phenyl-hexyl stationary phase. The rectangles indicate the saponin family QS-18 and QS-21 and between the lines the collected fraction corresponding to the first-pass semi-pure QS-21.

FIG. 11 depicts a chromatogram obtained for the Quillaja biomass extract, wherein chromatography was performed in the industrial liquid chromatography equipment using a 15 cm diameter DAC column filled with the 10 μm phenyl-hexyl stationary phase. The rectangles indicate the saponin family QS-18 and QS-21 and between the lines, the collected fraction corresponding to the first-pass semi-pure QS-21.

FIG. 12 depicts a quality control graph of first step of bark, wherein chromatography was used at analytical scale of the lyophilized powder to obtain in the first chromatographic step from quillaja bark extract, which was performed at an industrial scale. Using the sulfobetaine HILIC analytical column (sulfobetaine 10 μm, 250*4.6 mm column), the assignments of the main signals obtained are shown in boxes: QS-18, preceding peak, apiose isomer of QS-21 (QS-21 api), xylose isomer of QS-21 (QS-21 xyl).

FIG. 13 depicts second chromatographic step at the industrial scale of the lyophilized powder obtained in the first chromatographic step from Quillaja bark, showing the assignments of the main signals obtained in frames: QS-18, preceding peak, apiose isomer of QS-21 (QS-21 api), xylose isomer (QS-21 xyl). The fractions collected for each isomer of QS-21, QS-21 api and QS-21 xyl are shown between lines.

FIG. 14 depicts a second chromatographic step at the industrial scale of the lyophilized powder obtained in the first chromatographic step from Quillaja biomass, showing the assignments of the main signals obtained in frames: QS-18, preceding peak, apiose isomer of QS-21 (QS-21 Api), xylose isomer of QS-21 (QS-21 xyl). The fractions collected for each isomer of QS-21, QS-21 api and QS-21 xyl are shown between lines.

FIG. 15 depicts chromatographic quality control of the final QS-21 product obtained from Quillaja bark. The chromatographic purity and potency are close to 100% based on the area under the curve and mass quantified with respect to the commercial QS-21 standard.

FIG. 16 depicts chromatographic quality control of the final QS-21 product obtained from Quillaja biomass. It can be observed the presence of a majority peak, corresponding to QS-21, and two other minority peaks, corresponding to impurities.

FIG. 17 depicts comparison of chromatographic profile QS-21 obtained from bark and from biomass.

FIG. 18A depicts a chromatographic profile stretched in collection window over QS-21 peaks in the sulfobetaine HILIC column.

FIG. 18B depicts control quality of the QS-21 obtained by stretching the window.

FIG. 19 depicts antibody responses against the OVA antigen in mice vaccinated with different adjuvants.

FIG. 20 depicts OVA-specific CD4+ T cell proliferation response pulsed in vitro with OVA antigen; the group immunized with the xylose enriched formulation presented a greater proliferation of CD4+ T cells compared to the rest of the experimental groups.

FIG. 21 depicts correlation between T Cell proliferation (Normalized) with xylose content in QS-21 evaluated products (QS-21 75:25, QS-21 65:35, QS-21 apiose enriched 80:20 and QS-21 xylose enriched 3.3:96.7, V1:V2).

FIG. 21 shows a positive linear correlation found (R2=0.9973) between the % of xylose in the tested QS-21 and the % of T CD4 lymphocytes proliferation; without wishing to be bound by any particular theory, this indicates that the QS-21 xylose isomer has more activity in gathering the proliferation of the T CD4 lymphocytes.

FIG. 22 depicts percentage of OVA-specific CD4+/CD25+ T cells; the group immunized with the xylose enriched formulation presented a higher expression of the CD25 activation compared to the other experimental groups.

FIG. 23 depicts weight curves of mice immunized with the different treatments.

FIG. 24 depicts levels of antigen-specific antibodies against OVA measured by ELISA. One-way ANOVA with Bonferroni's multiple comparison test was used for statistical analyses (*p<0.05).

FIG. 25 depicts percentage of proliferation of CD4+ T cells from splenocytes of mice vaccinated with OVA (10 ug) antigen plus QS-21 adjuvant (16 μg) with varying apiose/xylose ratios. One-way ANOVA with Bonferroni's multiple comparison test was used for statistical analyses (*p<0.05).

FIG. 26 depicts correlation between T Cell proliferation (Normalized) with xylose content in six different isomeric QS-21 apiose:xylose proportion formulations.

FIG. 27 depicts comparison between normalized T Cell proliferation and QS-21 xylose content in formulations evaluated in the assays presented in Example 1 and Example 2.

FIG. 28 depicts percentage of OVA specific T Cell which express the Activation marker CD25 pulsed in vitro with OVA antigen. One-way ANOVA with Bonferroni's multiple comparison test was used for statistical analyses (*p<0.05).

FIG. 29 depicts weight % curve of each group of animals during the course of the immunization assay for Example 2.

FIG. 30 depicts measurement of total IgG RBD-specific antibody levels present in the sera of vaccinated mice of different groups.

FIG. 31 depicts percentage of proliferation of CD4+ T cells from splenocytes of mice vaccinated with 4 μg of RBD antigen plus QS-21 adjuvant (16 μg) with varying apiose:xylose ratios. One-way ANOVA with Bonferroni's multiple comparison test was used for statistical analyses (*p<0.05).

FIG. 32 depicts percentage of CD4+/CD25+ T cells from splenocytes of mice vaccinated with 4 μg of RBD antigen plus QS-21 adjuvant (16 μg) with varying apiose:xylose ratios.

FIG. 33 depicts weight % curve of each group of animals for Example 3; no significant weight loss was observed in the groups vaccinated with the isomeric formulations of QS-21 used in Example 3, and no toxic effects or significant behavioral changes were observed between the groups.

FIGS. 34A, 34B, 34C, 34D, 34E and 34F depict TICs of QS-21 obtained from standard QS-21 (75:25 V1:V2%) (FIG. 34A), QS-21 xylose (FIG. 34C) and QS-21 apiose (FIG. 34E). Signal zoomed between 1900-2000 Da for Standard QS-21 (FIG. 34B), QS-21 xylose (FIG. 34D) and QS-21 apiose (FIG. 34F). QS-21 xylose and QS-21 apiose with 100% isomeric purity respectively.

FIGS. 35A, 35B and 35C depict TICs of negative Multiple Ion Analysis (MI) of standard QS-21 (75:25%) of V1:V2 isomer (FIG. 35A), QS-21 xylose (0:100%) of V1:V2 isomer (FIG. 35B) and QS-21 apiose (100:0% of V1:V2 isomer (FIG. 35C), respectively. QS-21 (1988 Da [M−H]−) shows the same molecular ion pattern in the three products (m/z=1988/1; 249/8), noticeable is that m/z majoritarian corresponds to 249 Da, with denotes a z=8 in this methodology for all tested pure QS-21 products. Standard QS-21 (75:25%) of V1:V2 isomer.

FIGS. 36A, 36B and 36C depict HPLC analysis using the sulfobetaine analytical methodology for isomer determination of standard QS-21 (75:25%) of V1:V2 isomer (FIG. 36A), QS-21 apiose (100:0%) of V1:V2 isomer (FIG. 36B) and QS-21 xylose with 0:100% of V1:V2 isomer (FIG. 36C), respectively.

FIGS. 37A, 37B, 37C, and 37D depict analytical method for total saponin purity by HPLC using a C4 column. A 20 μL of solution of 2000 ppm for each sample was injected accordingly: Standard QS-21 (75:25) (FIG. 37A), QS-21-apiose (100:0) (FIG. 37B), QS-21-xylose (0:100) (FIG. 37C) Isomers and a Combination 1:1:1 in volume of each product (FIG. 37D).

### DESCRIPTION OF THE INVENTION

The present patent application describes a process for obtaining QS-21, wherein the QS-21 will have an apiose/xylose isomer ratio of less than 65:greater than 35 (apoise to xylose) (i.e., a xylose-rich QS-21) compared to that of standard QS-21, which contains at 65:35 V1:V2 (apiose:xylose ratio). More specifically, the modified QS-21 compounds will have a xylose-rich ratio ranging from 60:40 to 0.1:99.9 V1:V2 ratio, with at least some apoise isomer present. Said another way, the ratios can be set forth in percentages, wherein standard QS-21 and those products wishing to be provided in those same desired amounts will have 35% QS-21 xylose isomer and 65% apiose QS-21 isomer, while the modified QS-21 mixture of isomeric compounds will have from 40% to 99.9% xylose isomer compared to 60% and 0.1%, respectively, of the apiose isomer. In particular, the modified QS-21 mixture of isomeric compounds has unexpectedly been found to improve adjuvant activity as compared to the standard QS-21.

As either a part of the process above, or separately therefrom, the present patent application describes a process for obtaining QS-21 from different sources of the tree, Quillaja Saponaria. Further processes include the use and application of QS-21 and modified QS-21 mixture of isomeric compounds as a standalone immune adjuvant or in an immune adjuvant system with the same or improved adjuvanticity than the standard QS-21 or for use as a cancer therapeutic, which is only obtained from the bark of the Quillaja tree.

A further process that allows for the production of modified QS-21 enables the separation of the natural existing isomers of QS-21, namely, apiose and xylose, and thus obtain QS-21 from raw material, which may be bark, or may include the wood and the leaves, i.e., the entire biomass, and permits the production of modified QS-21 compounds and mixtures of isomers with different xylose/apiose ratios, ranged from 40 to 99.9% in xylose to 0.1 to 60% in apiose isomers.

Turning to the process that provides alternative sources other than bark for obtaining QS-21, it will be appreciated that the wood, the leaves, and the whole biomass are obtained by the pruning of the tree, wherein up to 30% of the total biomass can be obtained every five years without destroying the tree. Hence, this period allows the total regeneration of the specimen and strengthens tree growth. The abundance of this raw material and the development of the present patent application allows to obtain the same quantity of QS-21, but using only 2 acres of Quillaja forest, that would be fully regenerated in no more than five years period, which allows to overcome the supply problem of the prior art associated in the extraction of QS-21.

In one of the embodiments of the present application, a process to obtain QS-21 (saponins) from different sources of Quillaja is described, wherein said process comprises the following steps:
A) collect the raw material from the pruning of trees of the Genus Quillaja;
B) milling the raw material from the pruning of trees of the Genus Quillaja;
C) chipping the raw material from the pruning of trees of the Genus Quillaja;
D) providing an aqueous extract from the raw material of trees of the Genus Quillaja previously milled and chipped;
E) filtering the aqueous extract from step D);
F) concentrating the aqueous extract from step E);
G) clarification step to eliminate solids and purify the saponins from the extract of step F);
H) first and second filtering steps of the extract of step G) through press filters to produce a crude extract;
I) the crude extract of step H) is treated by ultrafiltration techniques increasing the saponin content up to 75-99% on a dry basis (ODB);
J) concentrate at vacuum the extract of step I), obtaining an ultra-pure saponin extract from the raw material of trees of the Genus Quillaja;
K) treating the ultra-pure extract of step J) with a High-Performance Liquid Chromatography (HPLC) separation with orthogonal two sequential steps, so as to obtain the QS-21 pure fraction as two separated peaks each corresponding to one of the QS-21 isomers, apiose or xylose.

It will be appreciated that step K) enables one to separate the apiose isomers and xylose isomers in different ratios. As a result of this method, modified QS-21 compounds, including novel mixtures of isomers, can be produced. One can setup the isomers ratio in the final QS-21 compounds by either displacement of the collection window of these two peaks; or by collecting each isomer as a different, completely separate fractions for later formulation at the desired isomers final ratio.

In one of the embodiments of the present application, in order to obtain a QS-21 pure fraction, a High-Performance Liquid Chromatography (HPLC) separation with Orthogonal two sequential steps is described and comprises the following steps:
1) a first chromatographic stage, comprising the use of reverse phase-based column:
1.1) collecting a QS-21 family fraction;
1.2) concentrate and organic solvent removal from the fraction obtained in 1.1) by;
1.2.1) nano-filtering the QS-21 family fraction of step 1.1);
1.2.2) Evaporate the organic solvent from the nanofiltrate of step 1.2.1);
1.3) proceeding with a vacuum drying of the fraction of step 1.2), obtaining the QS-21 impure fraction.
2) a second chromatographic stage, comprising a second HPLC step partition using a zwitterionic HILIC column under specific mobile phase conditions.
2.1) second HPLC step partition:
2.1.1) dissolving and filtering the QS-21 impure fraction collected from step 1.3) and use it as a feed material for the chromatography;
2.1.2) collect the fractions corresponding to QS-21 apiose and xylose. The fraction window collection can be adjusted by stretching it over the two peaks to increase purity and/or by displacement in the collection time; To the right to increase xylose and decrease apiose contents. To the left to increase apiose content and decrease xylose contents.
2.2) concentrate and organic solvent removal from the fraction obtained in 2.1.2) by evaporation.
2.3) proceeding with a freeze drying until total dryness, obtaining the QS-21 pure fraction.

In one of the embodiments of the present application, a QS-21 compound is described which is prepared from different sources of Quillaja according to the process described therein.

In one of the embodiments of the present application, a QS-21 compound is described which is used as a standalone immune adjuvant or in an immune adjuvant system for vaccines or as a cancer therapeutic.

The process allows to obtain QS-21 from raw material, the wood and the whole biomass in a less expensive way decreasing the production costs, sustainable means, increasing the production yields, and increasing the availability and supply of QS-21, enabling billions of doses for vaccines. This increases the available raw material, triggering an increase in production capacities, reducing costs and making the product sustainable, since the extraction of biomass does not cause the death of the tree or stress over the native Chilean forest. Also, the generated adjuvant product are analogs to the current QS-21 with less toxicity, more stability, better adjuvant activity, more versatility, and prolonged efficacy, and the product obtained by the process can be set up to provide for different isomer ratios giving a unique molecule fingerprint.

It will be appreciated that a fully sustainable process to obtain QS-21 from different sources of Quillaja, or that allows modified isomer ratios to provide either the standard 65:35 ratio of QS-21 found in the bark of the tree, or modified ratios of xylose-enriched or apiose enriched QS-21 products and their use as a standalone immune adjuvant or in an immune adjuvant system for vaccines or as a cancer therapeutic are important discoveries disclosed herein.

The present invention describes new QS-21 products obtained from different sources of Quillaja, that can have the same isomer ratio as that provided by standard QS-21 obtained from the bark of the tree. Those different sources may be the other parts of the tree. Where one uses the bark as well as the different other parts of the tree, such as the wood and leaves, it will be appreciated that this defines the “biomass” of the tree. Heretofore, no one has used the biomass of the Quillaja tree to produce QS-21 compounds, as everyone previously relied solely on the bark. Having additional parts of the tree for use, i.e., the biomass, allows for an improvement in the production of QS-21.

The present invention also describes new QS-21 products formulated with modified apiose and xylose ratios and the use of this new QS-21 isomers formulations with modified isomers ratio, ranging from 0.1-60% apiose and 40-99.9% xylose, wherein both isomers are necessarily present, as a standalone immune adjuvant or in an immune adjuvant system for vaccines or as a cancer therapeutic. It has been found that xylose-rich QS-21 compounds having an apiose to xylose isomer ratio ranging from 60:40 to 0.1:99.9 have better adjuvanticity and similar toxicity to standard QS-21.

Currently, Quillaja extracts for pharmaceutical use (mainly as immune enhancers) are obtained from the bark of the tree. This plant bark tissue is scarce and difficult to obtain, because it takes years to form and its extraction from the tree causes its death, which explains its high value. Therefore, alternatives to the use of Quillaja bark are being sought for the development of adjuvant products. In this context, the use of Quillaja biomass as a new generation of pharmaceutical products, such as adjuvants for use in animals and humans are being developed.

In the case of the adjuvant product for humans, efforts in this application are focused on the development of products of natural origin. In this context, QS-21 is considered a goldstandard and is already being used and administered in approved vaccines, for example against malaria. At present, the process of obtaining QS-21 originates only from bark as raw material. In this project, a two-step chromatographic method with orthogonality between them is developed. One advantage of this method is that it allows for obtaining QS-21 from any Quillaja raw material. The use of the tree's biomass, not just its bark, provides for more availability of the raw material, which, in turn reduces costs, is fully-sustainable, and has been found to not cause the death of the tree, which happens where only bark is used.

However, in providing such studies, it has been found that QS-21 compounds can actually be obtained that have different isomeric ratios than that customarily found in the bark of the tree. The example below provides a method for obtaining QS-21 from the biomass (i.e., the bark, wood and leaves). The resulting process enables one to separate the apiose and xylose isomers without maintaining the natural isomeric apiose:xylose ratio of 65:35 found in bark.

The following example is one method for obtaining QS-21 from Quillaja bark or biomass, with a purity higher than 90%. It will be appreciated that most of the steps denoted above have been already applied. This example essentially starts at step K) in the method above. Generally, the present invention provides for the use of High-Performance Liquid Chromatography (HPLC) with two, separate and independent steps, taking advantage of the orthogonality between reverse phase (RP) and hydrophilic interaction chromatography (HILIC) and/or chiral interaction columns. In the example, two powdered products, Q-Vax and Q-Vax-S (Q-Vant Biosciences), obtained from the bark and biomass respectively of the tree Quillaja saponaria Molina standardized to a high concentration of full-spectrum saponins and purity greater than 90%, were used as precursors. These products are designed to be utilized as a raw material for obtaining different fractions of saponins (i.e., QS-21, QS-7, etc.) and to facilitate their purification.

As a second step, an aqueous extract of the powdered product Q-Vax was tested through a reverse phase chromatography using columns of C4, C8, C18 C18 polar and both products on a phenyl-hexyl column. Once the resulting chromatography product was obtained, the resultant product was run through a second chromatographic step wherein HILIC columns were used with diol, amino and sulfobetaine. Chiral columns of cellucoat and aminocoat were also prepared and used. Those columns that produced the best results were studied at different pore sizes, at 7 and 10 μm, for their posterior scale-up to industrial scale. The products obtained from both liquid chromatography steps were freeze-dried at −50° C. for 5 days to obtain a powder.

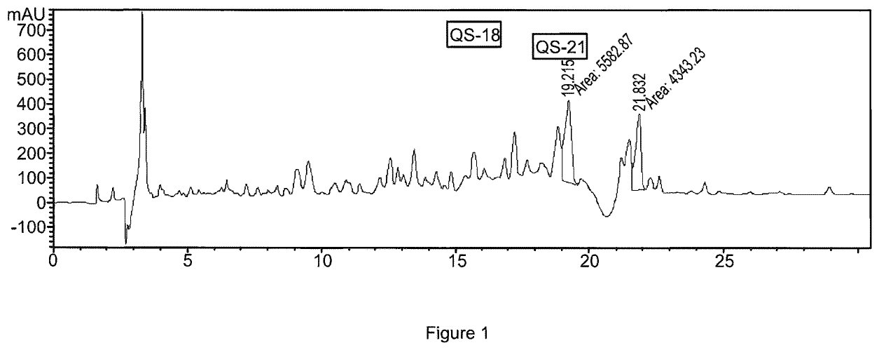

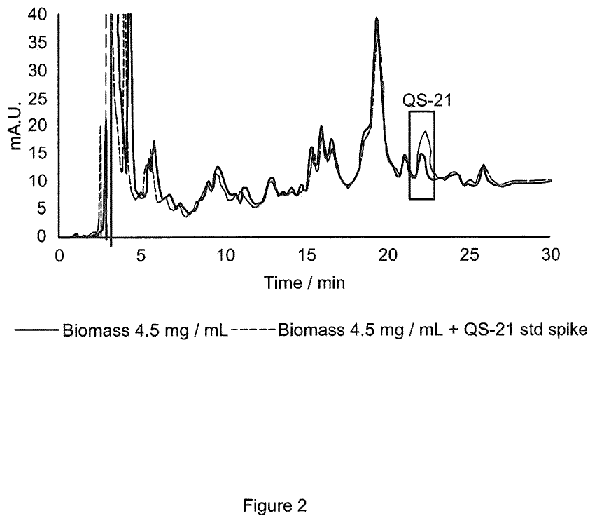

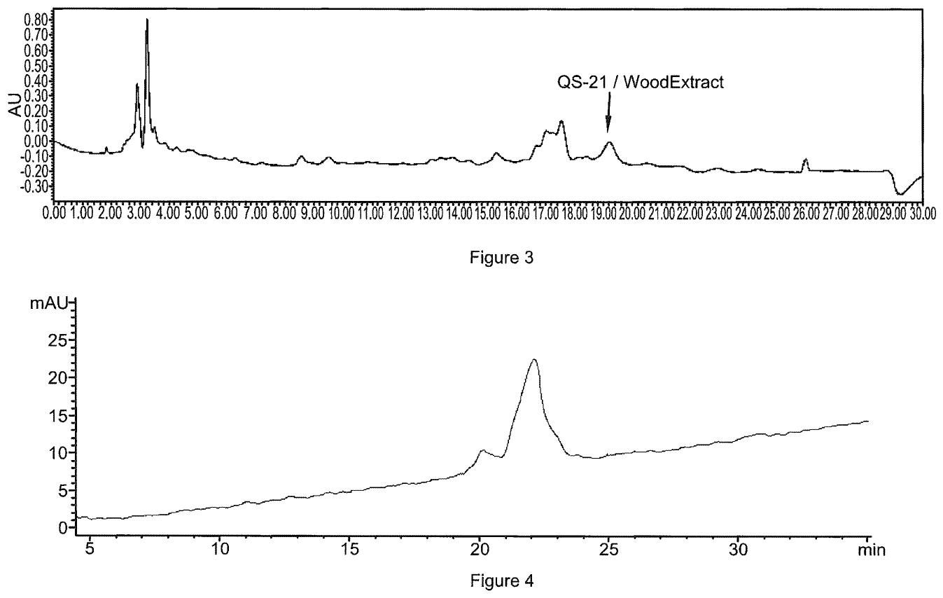

For the first chromatographic step, the separation between the saponin families present in the complete extracts obtained was evaluated. The RP type columns evaluated were C4, C8, C18, C18 polar and phenyl-hexyl. FIGS. 1-3 show the results obtained. FIG. 1 depicts a chromatographic profile of bark extract on a C18 column; difference in retention time between QS-18 and QS-21 of 3 minutes (using a water pulse). FIG. 2 depicts a chromatographic profile of Q-Vax S (biomass) on polar C18 column; difference in retention time between QS-18 and QS-21 of 1.6 minutes. FIG. 3 depicts a chromatographic profile of Q-Vax-S (biomass) extract on Phenyl-Hexyl column; difference in retention time between QS-18 and QS-21 of 2 minutes.

As can be seen in the previous images, with column C18 (FIG. 1) and using a water pulse (abrupt increase of the water content in the mobile phase) a separation of 3 minutes was achieved. This was the best separation obtained in terms of retention time, but the abrupt change in the composition of the mobile phase can cause damage to the stationary phase, so the study was continued with other columns, hoping to find one where the water pulse was not necessary. Subsequently, when using a polar C18 column (FIG. 2), the time separation between QS-18 and QS-21 was 1.6, minutes, this result was obtained without using water pulse. Finally, FIG. 3 shows the separation achieved by the Phenyl-Hexyl column, 2 minutes.

The C18 and C18 polar columns present better peak resolution than the Phenyl-Hexyl column, this is due to the fact that the pore size of the C18 columns is 5 μm, while for the Phenyl-Hexyl it is 10 μm. During the development of this study, the feasibility of scaling up the process was also evaluated. In conversations with suppliers of semi-preparative scale columns, it was concluded that it is not possible to work with 5 μm columns at a higher level than the analytical level, the available options range from 7 μm upwards. Therefore, it was decided that the scaling up of the first step should use a 10 μm Phenyl-Hexyl column.

The C4 column is the one normally used at analytical scale when working with saponins.

For the second chromatographic step, it is necessary to use columns capable of separating contaminants such as QS-18 and the preceding peak, which are the closest to QS-21 in the observed profiles. To achieve this, HILIC (diol, amino and sulfobetaine) and chiral (cellucoat and aminocoat) columns were tested.

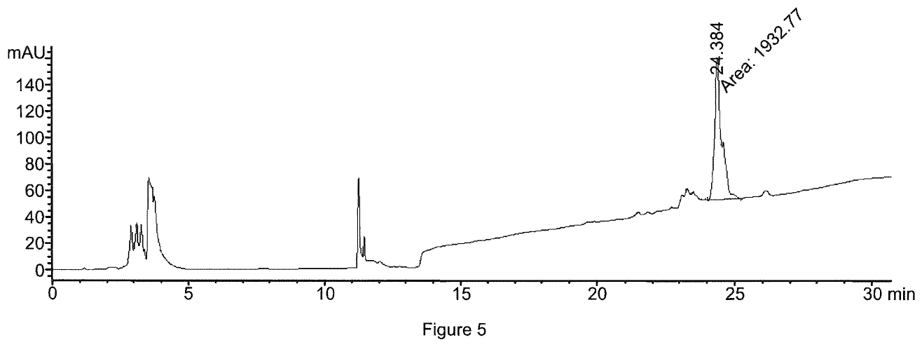

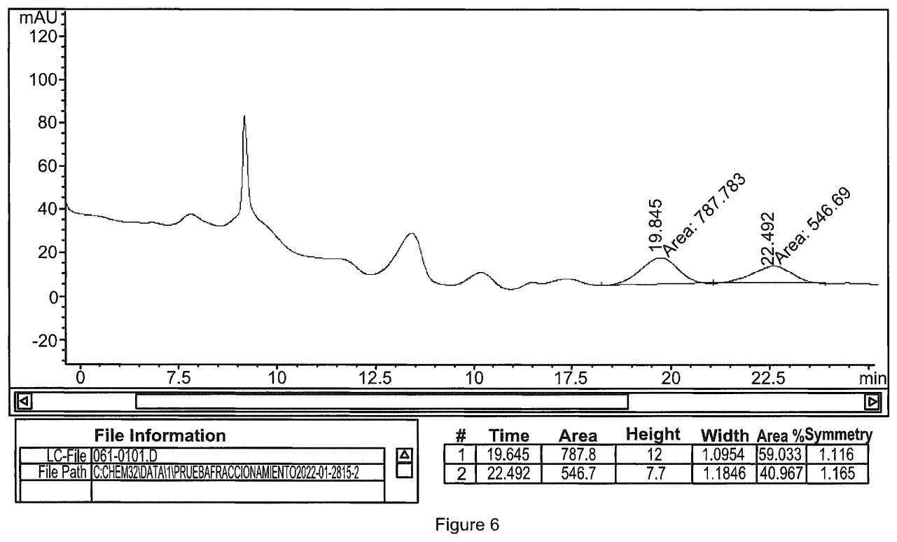

FIGS. 4-6 show some of the results obtained with the columns used in the second step. FIG. 4 depicts chromatographic profile second step in cellucoat column; the peak at 22 minutes corresponds to QS-21. FIG. 5 depicts chromatographic profile second step on diol column; the peak at 24 minutes corresponds to QS-21. FIG. 6 depicts chromatographic profile second step on sulfobetaine column 250×4.6 mm, 10 μm; peaks at 19.6 and 22.4 minutes correspond to isomers of QS-21 (apiose and xylose), Consistent results were not obtained with the diol column (FIG. 6), a peak was observed at 11 minutes that could not be identified, so this column was discarded. In the case of the cellucoat column (FIG. 4), a peak corresponding to QS-21 was obtained at minute 22, but the preceding peak at minute 20 elutes very close to QS-21. Finally, in the case of the sulfobetaine column (FIGS. 6 & 7), using an isocratic mobile phase of 84% ACN/16% water (5 mM ammonium acetate) with a flow at 1 mL/min and detection wavelength of 210 nm, not only was it possible to separate QS-21 from its contaminants, but also the ratio between these peaks suggest that both well separated peaks corresponds to the QS-21 apiose and xylose isomers respectively, resulting in an optimal separation of both isomers that has not been reported in the prior art. Considering the latter, using the sulfobetaine column, the separation allows for the obtention of high purity QS-21, in addition to controlling and modifying the apiose and xylose content present in the products, which naturally is approximately 65% apiose and 35% xylose, respectively according to the prior art¹. Therefore, the HILIC sulfobetaine column was chosen to scale up the second step.

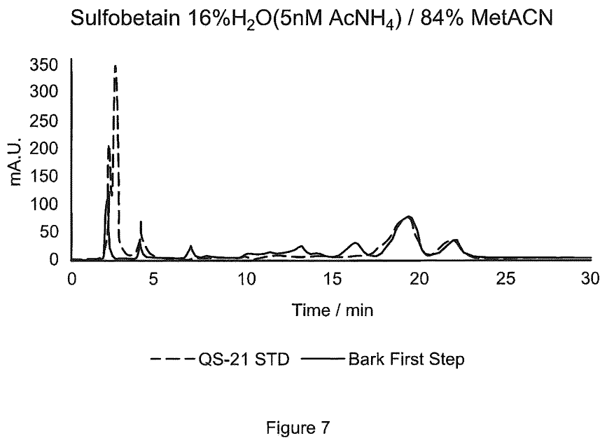

FIG. 7 depicts overlapped chromatogram of QS-21 std and impure QS-21 using sulfobetaine column; the QS-21 standard has two peaks in a ratio of 65:35 based in their relative AUC that correspond to the QS-21 isomers apiose (rT=19.8 min) and xylose (rT=22.1 min), respectively.

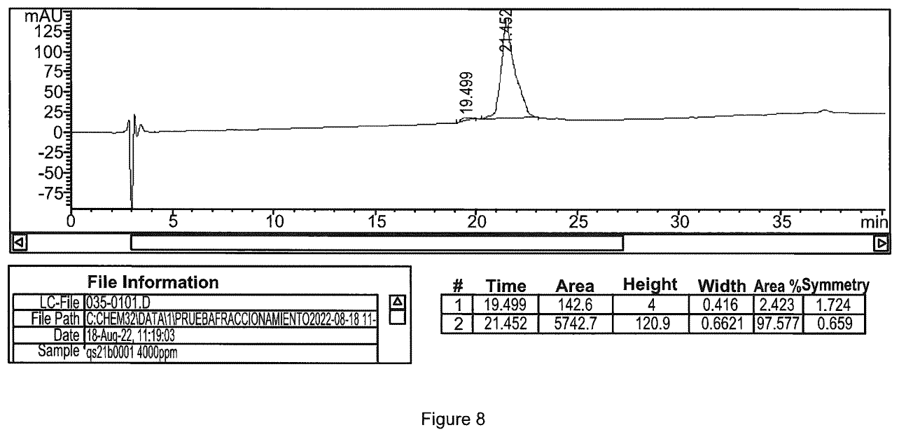

FIG. 8 depicts QS-21 quality control from biomass obtained from the second step; QS-21 represents 94.9% of the area. AUC that correspond to the QS-21 isomers apiose (rT=19.8 min) and xylose (rT=22.1 min), respectively. FIG. 8 is a C4 column DAD based HPLC QS-21 quality control chromatograph from biomass obtained from the second step. QS-21 represents 94.9% of the area.

In light of this discovery, it is possible to define a process to obtain QS-21, which delivers a product with a purity higher than 90%, even over 95%. For the industrial scale-up, the selected column was a Phenyl-Hexyl 10 μm column for use in RP chromatography for the first step and the HILIC sulfobetaine 10 μm column is used to perform the second step.

In light of the development of the analytical scale chromatographic methodologies above, the methodology to obtain and purify QS-21 was scaled up to a preparative scale. This process started by using a precursor with more than 90% of saponins in a 10 μm Phenyl-Hexyl column and 15 cm I.D. for the first chromatographic step, which allows for the separation of QS-18 and QS-21 from the rest of the saponin families, obtaining a semi-purified product of QS-21. Then, as a second chromatographic step, a HILIC sulfobetaine column of 10 μm and 5 cm I.D. is used to obtain a chromatographic orthogonality effect. With this, the objective of separating the saponins other than QS-21 that elute concomitantly (QS-18 and the preceding QS-21 peak), which are considered as impurities in obtaining QS-21, was achieved. Additionally, this HILIC column allows the separation of the QS-21 family into its two main isomers, QS-21 apiose, and QS-21 xylose. This, in itself, constitutes a new milestone since it is the first time that the efficient separation of these isomers has been achieved on an industrial scale, opening new commercial possibilities for the development of QS-21-based adjuvants with better immune activity and tolerance profiles compared to the standard QS-21 isomeric mixture. With this industrial-scale chromatographic process, one can obtain gram scale quantities of the purified QS-21 product separated into its isomers with a high degree of purity (>90%). Notably, the current dosing of QS-21 in licensed (approved vaccines) is between 25 to 50 micrograms.

To achieve the above, a linear scaling of the preferred conditions determined at the analytical scale was used. After obtaining the final products, analytical methodologies were implemented to be used as quality control to evaluate the purity of the products obtained by this process. The target compounds include (1) QS-21 with purity greater than 90% from biomass and bark, at industrial scale, with a similar isomer ratio to that of standard QS-21 (i.e., a 65:35 apiose:xylose ratio), (2) QS-21 with purity greater than 90% from biomass and bark, at industrial scale, with isomers ratio that is xylose-enriched (>40% of the isomeric composition, and, more particularly, at least 75%, 80% or 85% of the isomeric composition) and (3) QS-21 with purity greater than 90% from biomass and bark, at industrial scale, with isomers ratio that is apiose-enriched (>65% of the isomeric composition, and more particularly, at least 75% or 85% of the isomeric composition).

To demonstrate the practice of the invention, saponins were extracted from Quillaja trees. To do so, the Quillaja biomass (i.e., the bark, wood, and leaves) was pruned from the tree, and milled and chopped as previously indicated. An aqueous extract was prepared which was carried out at a mass/solvent ratio of 1:8 (biomass/bark: total mass). Water was used as solvent at 60° C., with agitation for 3 hours.

To eliminate contaminants and increase the concentration of saponins in the extract, tangential filtration techniques were used, using ultrafiltration membrane equipment, until a product with a saponin content of over 90% was obtained. The ultrafiltration was carried out at a temperature below 15° C., pressure of 5 bar, and maintaining a turbidity level below 100 NTU. After filtration, high-performance liquid chromatography (HPLC) was used for the quantification of the saponin content. A C4 column that has generally been used in work with saponins was used as the stationary phase. The mobile phase included water/TFA 0.15% and acetonitrile/TFA 0.15%.

Upon obtaining the necessary information regarding the saponin content, the aqueous extract was then used in the first chromatographic step at a preparative scale. A phenyl-hexyl column of 10 μm and 15 cm internal diameter was used as the stationary phase, while water/TFA 0.15% and acetonitrile/TFA 0.15% were used as the mobile phase. For the case of bark, the total solid loading of the precursor on the column was 19.2 g per injection. While for biomass the solid loading of the precursor on the column was 14.4 g per injection.

To increase the concentration of saponins and decrease the solvent volume of the first chromatographic collection, a tangential nanofiltration technique was used, using membrane equipment at a temperature of 15° C. and a pressure of 5 bar. The product obtained was subjected to rotary evaporation using conventional rotavapor equipment and subsequently lyophilized (freeze-dried) and was used as the starting material for the second chromatographic step.

The second chromatographic step was then conducted at a preparative scale. To do so, a HILIC sulfobetaine column of 10 μm and 5 cm internal diameter and 25 cm length was used as the stationary phase, with water, ammonium acetate (5 mM), and acetonitrile being used as the mobile phase in isocratic conditions ranging ACN from 75% to 90%, more preferable 80% to 85%, and even more preferably at 82-83% ACN. For the case of bark, the total solids loading of the freeze-dried product (obtained from the first step) on the column was 90 mg per injection, while for the biomass the loading of solids in the column was 150 mg per injection. During this chromatographic partitioning step, the fractions corresponding to QS-21 apiose and QS-21 xylose were collected.

After each chromatographic step, the product obtained was taken to a rotary evaporator to evaporate the organic solvent used in each mobile phase. The conditions were 30° cat 40 rpm. The product obtained in water was freeze-dried under high vacuum conditions for 4 days until a freeze-dried powder was obtained.

The powder obtained was quantified for purity by HPLC, using a C4 column. A calibration curve was performed using QS-21 obtained from bark with a 65:35 apiose:xylose ratio as a standard. Area and mass percentages were compared.

The isomeric ratio of the resultant QS-21 powders was then determined. The QS-21 isomers corresponding fractions and the final products were characterized in their isomeric ratio using a sulfobetaine HILIC analytical column under isocratic conditions where the area of the peaks corresponding to apiose or xylose isomers were quantified in their percentage (%) of the total area.

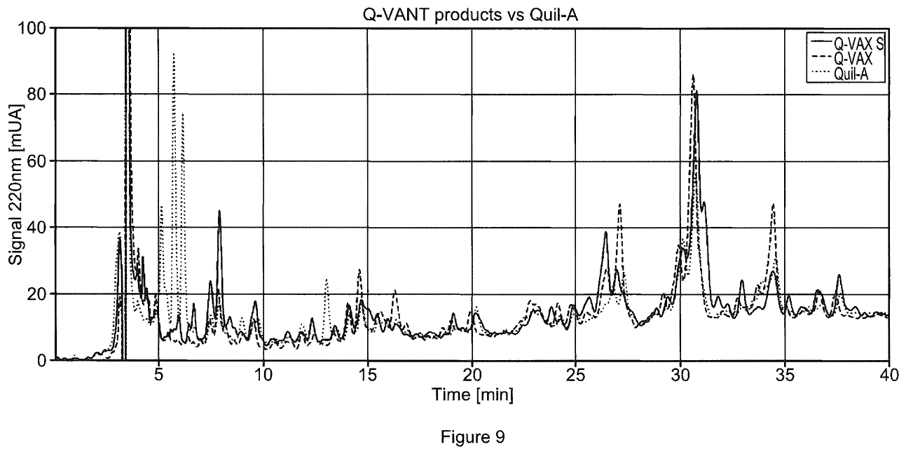

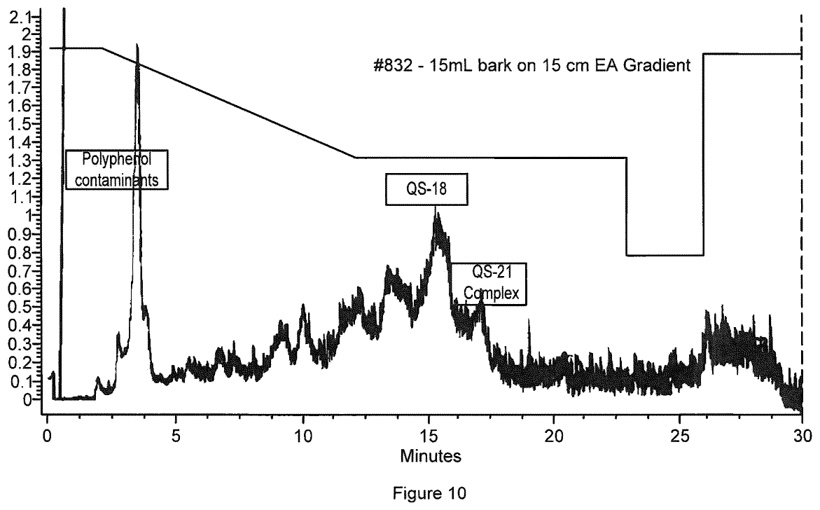

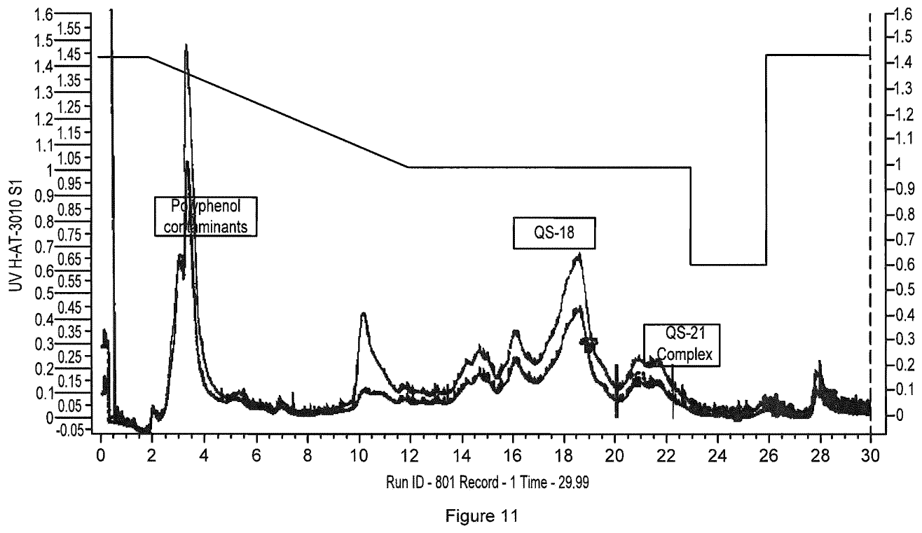

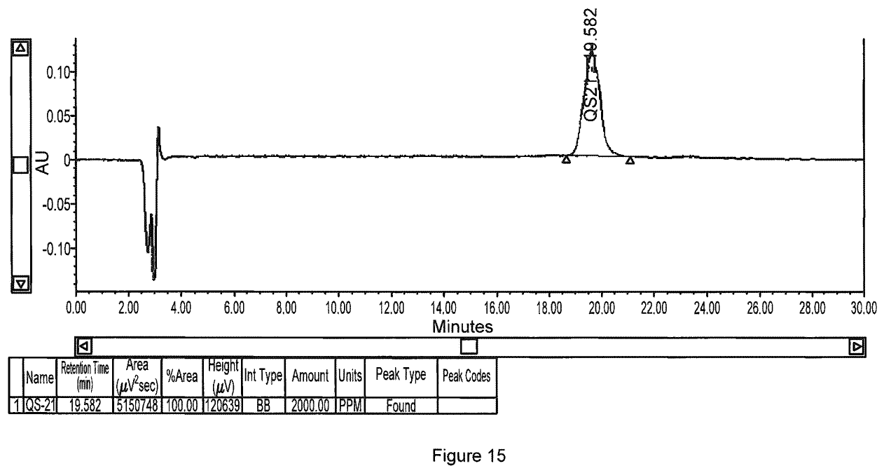

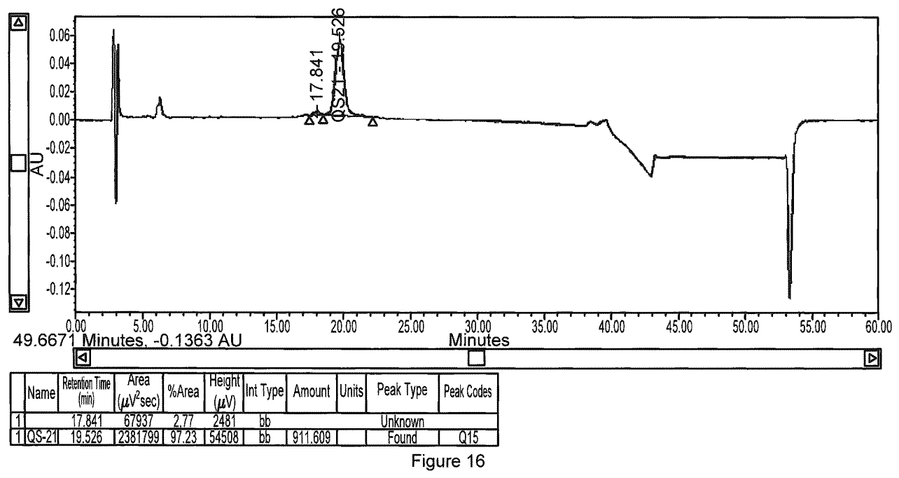

From the precursors with a saponin content above 90%, whose chromatographic profile is shown in FIG. 9, the first chromatographic step at the preparative scale detailed in this description was initiated. The chromatograms obtained for both bark and biomass at the preparative level are shown in FIGS. 15 and 16, respectively. For the case of bark, 10 injections were performed, and for the case of biomass, 20 injections were performed. FIG. 9 depicts comparison on saponin profile of Q-VANT extracts obtained from bark (Q-Vax), biomass (Q-Vax-S) and Quil-A (Croda International, plc product). FIG. 10 depicts chromatogram obtained for the Quillaja bark extract. Chromatography was performed in the industrial liquid chromatography equipment using a 15 cm diameter DAC column filled with 10 μm phenyl-hexyl stationary phase. The rectangles indicate the saponin family QS-18 and QS-21 and between the lines the collected fraction corresponding to the first-pass semi-pure QS-21. FIG. 11 depicts chromatogram obtained for the Quillaja biomass extract. Chromatography was performed in the industrial liquid chromatography equipment using a 15 cm diameter DAC column filled with the 10 μm phenyl-hexyl stationary phase. The rectangles indicate the saponin family QS-18 and QS-21 and between the lines, the collected fraction corresponding to the first-pass semi-pure QS-21.

The chromatogram obtained for the bark extract (FIG. 10) in the first step shows an overlap of the QS-18 peak with the base of the QS-21 peak, which means that a semi-pure QS-21 fraction was obtained. This fraction possessed a decreased, but not zero, content of QS-18 along with QS-21. However, the column did successfully separate QS-21 from the rest of the saponin families (QS-7 and QS-17).

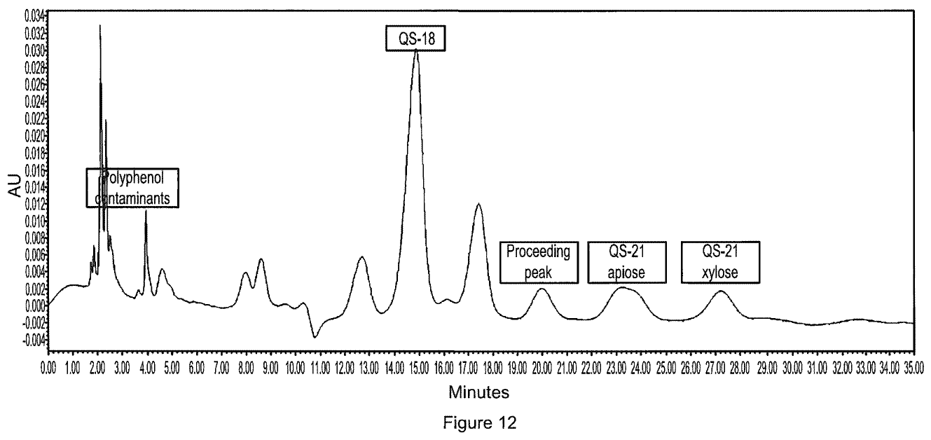

In the case of biomass (FIG. 11), it can be observed that there was an overlap of peak QS-18 with peak QS-21 and that the latter is of lesser magnitude than in the bark, given by QS-21 lower abundance in this raw material. Although this implies a greater difficulty in the separation, it was possible to obtain a semi-pure fraction with a high content of QS-21 in relation to QS-18. To definitively purify QS-21 and eliminate the remaining QS-18, both for bark (FIG. 10) and biomass (FIG. 11), the fractions obtained in the first step were freeze-dried (lyophilized). The powder obtained (QS-21 semi-pure) from bark and biomass was analyzed at an analytical scale using the same type of column as the second step (10 μm sulfobetaine), as a process control. The process control performed at analytical scale is shown in FIG. 12 and a complete separation between QS-21, QS-18, preceding peak and other additional peaks, which the column used in the first step is not able to separate, can be observed. In addition, this column also achieved the separation of the QS-21 isomers apiose and xylose. With this result, a second step at the industrial scale was performed.

FIG. 12 depicts quality control first step of bark. Chromatography at analytical scale of the lyophilized powder obtained in the first chromatographic step from quillaja bark extract was performed at an industrial scale. Using the sulfobetaine HILIC analytical column (sulfobetaine 10 μm, 250*4.6 mm column). The assignments of the main signals obtained are shown in boxes: QS-18, preceding peak, apiose isomer of QS-21 (QS-21 api), xylose isomer of QS-21 (QS-21 xyl).

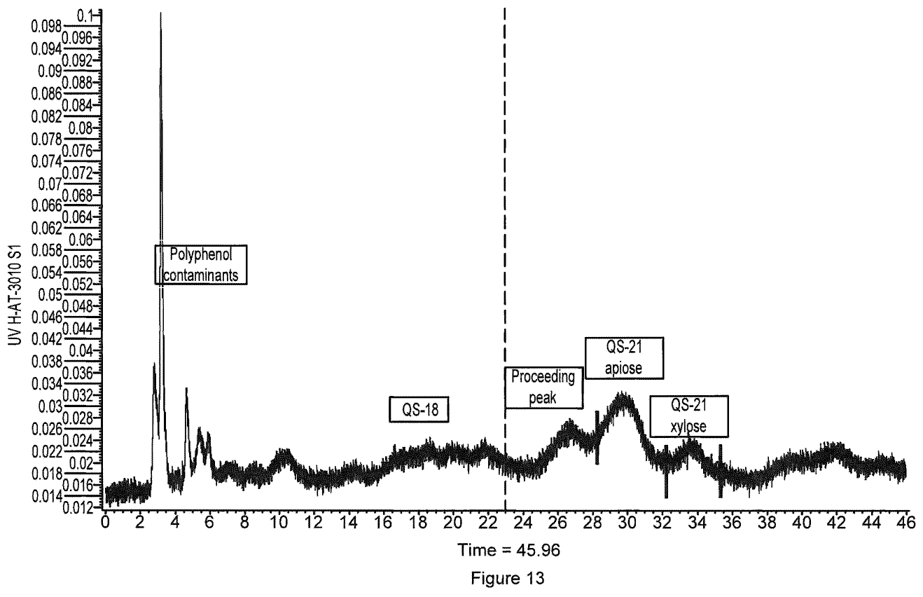

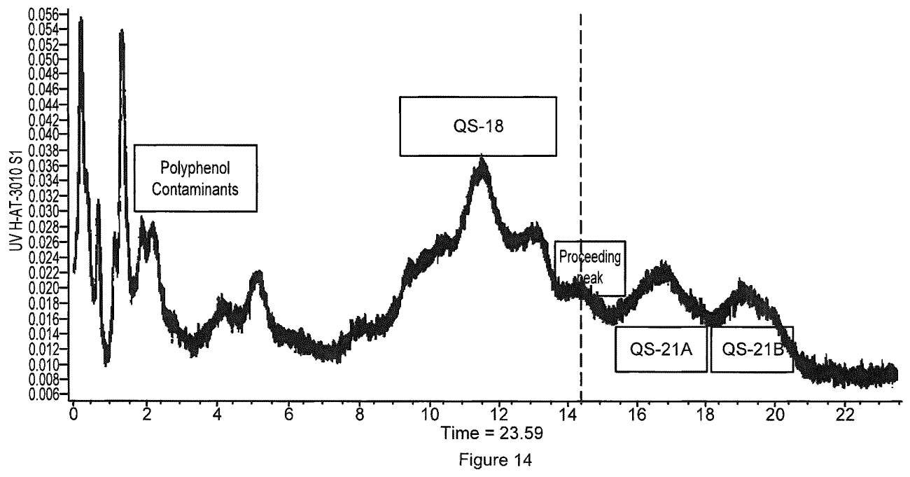

In the second chromatographic step at the preparative scale, a 10 μm 5 cm I.D. sulfobetaine HILIC column was used to obtain a chromatographic orthogonality effect. As can be seen in FIG. 13 for bark and FIG. 14 for biomass, the second chromatographic step at the preparative level achieved the objective of separating the saponins other than QS-21 that elute concomitantly (QS-18 and preceding peak), which are considered as impurities in obtaining QS-21. Additionally, this preparative column, as well as the analytical scale column of the same type, allowed the separation of the QS-21 family into its two main isomers, QS-21apiose (V1) and QS-21 xylose V2.

FIG. 13 depicts the second chromatographic step at the industrial scale of the lyophilized powder obtained in the first chromatographic step from Quillaja bark. The assignments of the main signals obtained are shown in frames: QS-18, preceding peak, apiose isomer of QS-21 (QS-21 api), xylose isomer (QS-21 xyl). The fractions collected for each isomer of QS-21, QS-21 api and QS-21 xyl are shown between lines.

FIG. 14 depicts the second chromatographic step at the industrial scale of the lyophilized powder obtained in the first chromatographic step from Quillaja biomass. The assignments of the main signals obtained are shown in frames: QS-18, preceding peak, apiose isomer of QS-21 (QS-21 Api), xylose isomer of QS-21 (QS-21 xyl). The fractions collected for each isomer of QS-21, QS-21 apiose and QS-21 xylose are shown between lines.

Finally, the products of the second chromatographic step were freeze-dried to obtain a lyophilized powder as the final product. The final products of QS-21 and its isomers, obtained from both bark and biomass were subjected to quality control using the HPLC methodology described by San Martin and Briones (San Martin & Briones, J Sci Food Agric 80:2063-2068, 2000), the disclosure of which is incorporated herein by reference, to quantify the purity of QS-21 in the final products. QS-21 from bark in a 65:35 apiose:xylose ratio was used as a standard.

FIG. 15 depicts chromatographic quality control of the final QS-21 product obtained from Quillaja bark. The chromatographic purity and potency are close to 100% based on the area under the curve and mass quantified with respect to the commercial QS-21 standard. In the case of the bark, FIG. 15 shows the quality control chromatogram. From this, it can be seen that the chromatographic and mass % purity is close to 100% based on the area under the curve with respect to the bark extract QS-21 standard.

FIG. 16 depicts chromatographic quality control of the final QS-21 product obtained from Quillaja biomass. It can be observed the presence of a majority peak, corresponding to QS-21, and two other minority peaks, corresponding to impurities. From this it can be seen that the chromatographic purity of QS-21 is 95% (first arrow) based on the area under the total curve of the chromatogram.

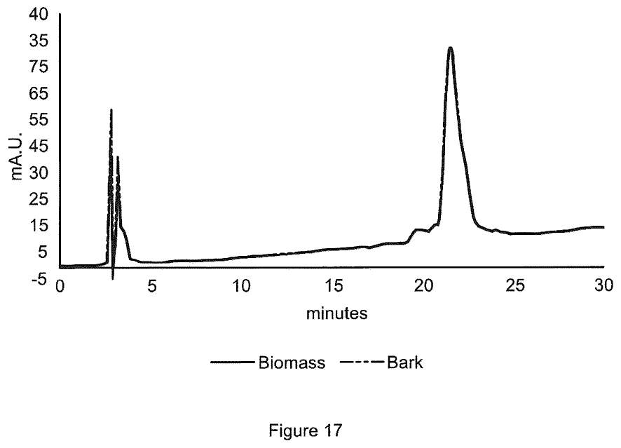

Also, in FIG. 17 is presented a QC HPLC chromatogram performed using a C4 column accordingly to the method described by San Martin & Briones (2000) where both QS-21 products, bark and biomass obtained are soluble in water/acetonitrile at 1000 ppm solution. Both samples were filtered and subsequently, they were injected into the HPLC using the C4 column, to quantify and compare the QS-21 content present in both samples.

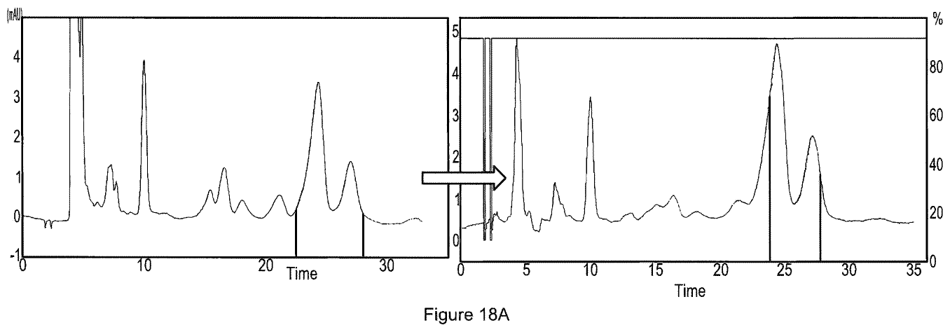

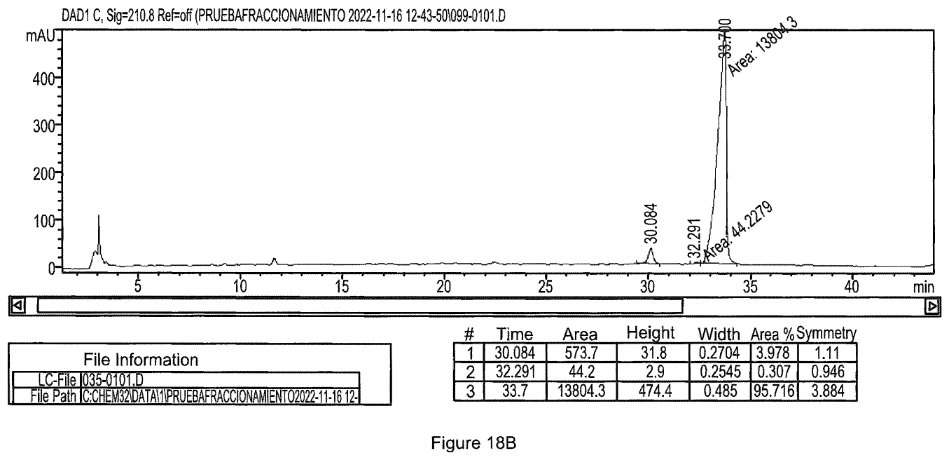

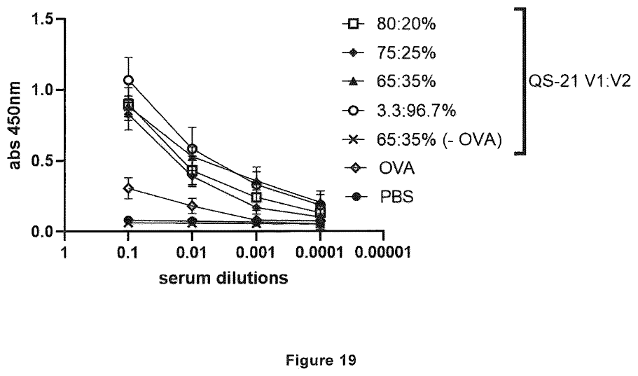

From FIG. 17, it is observed that the retention time and concentration of QS-21 in both cases is the same, indicating that the QS-21 obtained is the same using bark or biomass as starting raw material. FIG. 18A depicts stretched in collection window over QS-21 peaks in the sulfobetaine HILIC column. FIG. 18B depicts control quality of the QS-21 obtained by stretching the window.

In light of the foregoing, it was possible to obtain a QS-21 product with purity and potency higher than 90% from the bark and wood biomass. Stretching the window collection of the HILIC chromatographic products with a purity higher than 95% QS-21 purity was obtained. Further, modifying the collection with displacement to the right in the QS-21 peaks in the HILIC chromatography gives a product with an increased xylose isomeric ratio. This increase ranged from 40 to up to 99.9% in xylose content depending on the displacement among the apiose/xylose peaks and modifying the collection with displacement to the left in the QS-21 peaks in the chromatogram gives a product with an increased apiose isomeric ratio. This increase ranged from 40 to up 99.9% in apiose content depending on the displacement over the apiose/xylose peaks (FIG. 18).

Collection of the isolated peak for apiose and xylose allows to obtain QS-21 products with modified isomeric ratios from up to 95%, but at least 85% in each case (xylose enriched or apiose enriched). Also, by previous standardization of the isomeric ratio in each of these fractions, through analytic sulfobetaine HILIC column mixes can be performed. This allows to formulate new QS-21 products where the ratio can be set up from 40-99.9% xylose to 0.1-60% % apiose, which maintains the purity over 95% of QS-21, but with modified isomeric composition regarding the traditional bark obtained QS-21.

The invention provides a modified QS-21 mixture of isomer compounds used as a standalone immune adjuvant or in an immune adjuvant system or as a cancer therapeutic, wherein the QS-21 compounds comprise: apiose (V1) isomers and xylose (V2) isomers in a ratio ranging from about 60:40 to 0.1:99.9, wherein the modified QS-21 mixture of isomer compounds provides greater adjuvanticity and no difference in toxicity as compared to a standard QS-21 compound containing apiose (V1) isomers and xylose (V2) isomers in a ratio of 65:35. In some embodiments, the apiose to xylose ratio is selected from 60:40, 59:41, 58:42, 57:43, 56:44, 55:45, 54:46, 53:47, 52:48, 51:49, 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, 1:99, and 0.1:99.9. In some embodiments, the disclosure provides the modified QS-21 mixture of isomer compounds as described herein, wherein the ratio of apiose (V1) isomers to xylose (V2) isomers is about 20:80, while in other embodiments, the ratio is about 25:75, and still other embodiments, it is about 30:70. In some embodiments, the disclosure provides the modified QS-21 mixture of isomer compounds disclosed herein, wherein the saponin is taken from the bark and at least one other part of Quillaja plant. In some embodiments, the disclosure provides a modified QS-21 mixture of isomer compounds comprising apiose-enriched isomers, wherein the apiose (V1) isomers and xylose (V2) isomers have a ratio ranging from 70:30 to 99.9:0.1. In some embodiments, the apiose to xylose ratio is selected from 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, and 99.9:0.1. In some embodiments, the disclosure provides the use of a modified QS-21 mixture of isomer compounds described herein as an immune adjuvant.

The disclosure also provides a process for obtaining QS-21 from different sources of Quillaja and their use as a standalone immune adjuvant or in an immune adjuvant system or as a cancer therapeutic, wherein said process comprises one or more steps selected from: A) collect the raw material from the pruning of trees of the Genus Quillaja; B) milling the raw material from the pruning of trees of the Genus Quillaja; C) chipping the raw material from the pruning of trees of the Genus Quillaja; D) providing an aqueous extract from the raw material of trees of the Genus Quillaja previously milled and chipped; E) filtering the aqueous extract from step D); F) concentrating the aqueous extract from step E); G) clarification step to eliminate solids and purify the saponins from the extract of step F); H) first and second filtering steps of the extract of step G) through press filters to produce a crude extract; I) the crude extract of step H) is treated by ultrafiltration techniques increasing the saponin content up to 75-99% on a dry basis (ODB); J) concentrate at vacuum the extract of step I), obtaining an ultra-pure saponin extract from the raw material of trees of the Genus Quillaja; and K) treating the ultra-pure extract of step J) with a High-Performance Liquid Chromatography (HPLC) separation with Orthogonal two sequential steps, obtaining a QS-21 pure fraction.

In some embodiments, the disclosure provides a QS-21 saponin that is xylose-enriched and purified from a crude Quillaja saponaria extract, wherein the pure saponin is characterized by essentially a single predominant peak comprising 90% or more of the total area of all peaks of a chromatogram, excluding the solvent peak, when analyzed on a hydrophilic interaction liquid chromatography (HILIC) using sulfobetaine 250×4.6 mm, 10 μm, 100 Å under isocratic conditions of a mixture comprising 84% ACN/16% water (5 mM ammonium acetate) at 1 mL min-1 flow rate and 210 nm of wavelength detection. In some embodiments, the essentially single predominant peak comprises 80% or more of the total area of all peaks, 81% or more of the total area of all peaks, 82% or more of the total area of all peaks, 83% or more of the total area of all peaks, 84% or more of the total area of all peaks, 85% or more of the total area of all peaks, 86% or more of the total area of all peaks, 87% or more of the total area of all peaks, 88% or more of the total area of all peaks, 89% or more of the total area of all peaks, 90% or more of the total area of all peaks, 91% or more of the total area of all peaks, 92% or more of the total area of all peaks, 93% or more of the total area of all peaks, 94% or more of the total area of all peaks, 95% or more of the total area of all peaks, 96% or more of the total area of all peaks, 97% or more of the total area of all peaks, 98% or more of the total area of all peaks, or 99% or more of the total area of all peaks. The term “essentially” is used to describe the peak to note that while there is only one predominant peak, there still exists a second peak related to the apoise isomer. However, the peak for the xylose-enriched saponin is so significantly great as to be, in essence, a single peak. In some embodiments, a substantially pure QS-21 xylose-enriched saponin isomer disclosed herein is characterized by an essentially single predominant peak comprising 80-85% of the total area of all peaks of the chromatogram, 85-90% of the total area of all peaks of the chromatogram, 90-95% of the total area of all peaks of the chromatogram, or 95-99% of the total area of all peaks of the chromatogram. In some embodiments, a substantially pure QS-21 xylose-enriched saponin isomer disclosed herein is characterized by an essentially single predominant peak comprising at least 95%, at least 96%, at least 97%, at least 98%, or at least 99% of the total area of all peaks of the chromatogram. The disclosure also provides an immunogenic composition comprising an adjuvant composition comprising a substantially pure QS-21 xylose-enriched saponin isomer disclosed herein, and an immunogen or antigen, or a polynucleotide encoding the immunogen or antigen.

The disclosure provides an immunogenic composition comprising: an adjuvant composition comprising a QS-21 xylose-enriched saponin isomer purified from a crude Quillaja saponaria extract, wherein the saponin is characterized by an essentially single predominant peak comprising 90% or more of the total area of all peaks of a chromatogram, excluding the solvent peak, when analyzed on a hydrophilic interaction liquid chromatography (HILIC) using sulfobetaine 250×4.6 mm, 10 μm, 100 Å under Isocratic conditions of a mixture comprising 84% ACN/16% water (5 mM ammonium acetate) at 1 mL min-1 flow rate and 210 nm of wavelength detection; and an immunogen or antigen, or a polynucleotide encoding the immunogen or antigen. In some embodiments, the essentially single predominant peak comprises 80% or more of the total area of all peaks, 81% or more of the total area of all peaks, 82% or more of the total area of all peaks, 83% or more of the total area of all peaks, 84% or more of the total area of all peaks, 85% or more of the total area of all peaks, 86% or more of the total area of all peaks, 87% or more of the total area of all peaks, 88% or more of the total area of all peaks, 89% or more of the total area of all peaks, 90% or more of the total area of all peaks, 91% or more of the total area of all peaks, 92% or more of the total area of all peaks, 93% or more of the total area of all peaks, 94% or more of the total area of all peaks, 95% or more of the total area of all peaks, 96% or more of the total area of all peaks, 97% or more of the total area of all peaks, 98% or more of the total area of all peaks, or 99% or more of the total area of all peaks.

More particularly, the invention also provides a substantially pure QS-21 saponin composition purified from a crude Quillaja saponaria extract, the composition comprising a QS-21 apiose saponin isomer and a QS-21 xylose saponin isomer in a ratio of (A parts apiose isomer):(X parts xylose isomer), wherein: 0.1<A<65; 35<X<99.9, where A+X=100; and the pure saponin composition is characterized by one or more predominant peaks comprising 90% or more of the total area of all peaks of a chromatogram, excluding the solvent peak, when analyzed on a hydrophilic interaction liquid chromatography (HILIC) using sulfobetaine 250×4.6 mm, 10 μm, 100 Å under Isocratic conditions of a mixture comprising 84% ACN/16% water (5 mM ammonium acetate) at 1 mL min-1 flow rate and 210 nm of wavelength detection. In some embodiments, A:X is selected from 60:40, 59:41, 58:42, 57:43, 56:44, 55:45, 54:46, 53:47, 52:48, 51:49, 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, 1:99, and 0.1:99.9. In some embodiments, the one or more predominant peaks comprise 80% or more of the total area of all peaks, 81% or more of the total area of all peaks, 82% or more of the total area of all peaks, 83% or more of the total area of all peaks, 84% or more of the total area of all peaks, 85% or more of the total area of all peaks, 86% or more of the total area of all peaks, 87% or more of the total area of all peaks, 88% or more of the total area of all peaks, 89% or more of the total area of all peaks, 90% or more of the total area of all peaks, 91% or more of the total area of all peaks, 92% or more of the total area of all peaks, 93% or more of the total area of all peaks, 94% or more of the total area of all peaks, 95% or more of the total area of all peaks, 96% or more of the total area of all peaks, 97% or more of the total area of all peaks, 98% or more of the total area of all peaks, or 99% or more of the total area of all peaks. In some embodiments, a substantially pure QS-21 saponin composition described herein is characterized by a single predominant peak comprising 90-95% of the total area of all peaks of the chromatogram. In some embodiments, a substantially pure QS-21 saponin composition described herein is characterized by a single predominant peak comprising at least 95% of the total area of all peaks of the chromatogram. In some embodiments, the disclosure provides an immunogenic composition comprising an adjuvant composition comprising a substantially pure QS-21 saponin composition disclosed herein, and an immunogen or antigen, or a polynucleotide encoding the immunogen or antigen.

The disclosure also provides an immunogenic composition comprising: an adjuvant composition comprising a substantially pure QS-21 saponin composition purified from a crude Quillaja saponaria extract, the composition comprising a QS-21 apiose saponin isomer and a QS-21 xylose saponin isomer in a ratio of (A parts apiose isomer):(X parts xylose isomer), wherein: 0.1<A<65; 35<X<99.9, wherein A+X=100; and the pure saponin composition is characterized by one or more predominant peaks comprising 90% or more of the total area of all peaks of a chromatogram, excluding the solvent peak, when analyzed on a hydrophilic interaction liquid chromatography (HILIC) using sulfobetaine 250×4.6 mm, 10 μm, 100 Å under isocratic conditions of a mixture comprising 84% ACN/16% water (5 mM ammonium acetate) at 1 mL min-1 flow rate and 210 nm of wavelength detection; and an immunogen or antigen, or a polynucleotide encoding the immunogen or antigen. In some embodiments, A:X is selected from 60:40, 59:41, 58:42, 57:43, 56:44, 55:45, 54:46, 53:47, 52:48, 51:49, 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, 1:99, and 0.1:99.9. In some embodiments, the one or more predominant peaks comprise 80% or more of the total area of all peaks, 81% or more of the total area of all peaks, 82% or more of the total area of all peaks, 83% or more of the total area of all peaks, 84% or more of the total area of all peaks, 85% or more of the total area of all peaks, 86% or more of the total area of all peaks, 87% or more of the total area of all peaks, 88% or more of the total area of all peaks, 89% or more of the total area of all peaks, 90% or more of the total area of all peaks, 91% or more of the total area of all peaks, 92% or more of the total area of all peaks, 93% or more of the total area of all peaks, 94% or more of the total area of all peaks, 95% or more of the total area of all peaks, 96% or more of the total area of all peaks, 97% or more of the total area of all peaks, 98% or more of the total area of all peaks, or 99% or more of the total area of all peaks.

The disclosure also provides a substantially pure QS-21 saponin composition purified from a crude Quillaja saponaria extract, the composition comprising a QS-21 apiose saponin isomer and a QS-21 xylose saponin isomer in a ratio of (A parts apiose isomer):(X parts xylose isomer), wherein: 65<A<99.9; 0.1<X<35, wherein A+X=100; and the pure saponin composition is characterized by one or more predominant peaks comprising 90% or more of the total area of all peaks of a chromatogram, excluding the solvent peak, when analyzed on a hydrophilic interaction liquid chromatography (HILIC) using sulfobetaine 250×4.6 mm, 10 μm, 100 Å under Isocratic conditions of a mixture comprising 84% ACN/16% water (5 mM ammonium acetate) at 1 mL min-1 flow rate and 210 nm of wavelength detection. In some embodiments, A:X is selected from 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, and 99/9:0.1. In some embodiments, the one or more predominant peaks comprise 80% or more of the total area of all peaks, 81% or more of the total area of all peaks, 82% or more of the total area of all peaks, 83% or more of the total area of all peaks, 84% or more of the total area of all peaks, 85% or more of the total area of all peaks, 86% or more of the total area of all peaks, 87% or more of the total area of all peaks, 88% or more of the total area of all peaks, 89% or more of the total area of all peaks, 90% or more of the total area of all peaks, 91% or more of the total area of all peaks, 92% or more of the total area of all peaks, 93% or more of the total area of all peaks, 94% or more of the total area of all peaks, 95% or more of the total area of all peaks, 96% or more of the total area of all peaks, 97% or more of the total area of all peaks, 98% or more of the total area of all peaks, or 99% or more of the total area of all peaks. In some embodiments, a substantially pure QS-21 saponin composition described herein is characterized by a single predominant peak comprising 90-95% of the total area of all peaks of the chromatogram. In some embodiments, a substantially pure QS-21 saponin composition of claim 22, characterized by a single predominant peak comprising at least 95% of the total area of all peaks of the chromatogram. In some embodiments, the disclosure also provides an immunogenic composition comprising an adjuvant composition comprising a substantially pure QS-21 saponin composition described herein, and an immunogen or antigen, or a polynucleotide encoding the immunogen or antigen.

The disclosure also provides an immunogenic composition comprising: an adjuvant composition comprising a substantially pure QS-21 saponin composition purified from a crude Quillaja saponaria extract, the composition comprising a QS-21 apiose saponin isomer and a QS-21 xylose saponin isomer in a ratio of (A parts apiose isomer):(X parts xylose isomer), wherein: 65<A<99.9; 0.1<X<35, wherein A+X=100; and the pure saponin composition is characterized by one or more predominant peaks comprising 90% or more of the total area of all peaks of a chromatogram, excluding the solvent peak, when analyzed on a hydrophilic interaction liquid chromatography (HILIC) using sulfobetaine 250×4.6 mm, 10 μm, 100 Å under Isocratic conditions of a mixture comprising 84% ACN/16% water (5 mM ammonium acetate) at 1 mL min-1 flow rate and 210 nm of wavelength detection; and an immunogen or antigen, or a polynucleotide encoding the immunogen or antigen. In some embodiments, A:X is selected from 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, and 99.9:0.1. In some embodiments, the one or more predominant peaks comprise 80% or more of the total area of all peaks, 81% or more of the total area of all peaks, 82% or more of the total area of all peaks, 83% or more of the total area of all peaks, 84% or more of the total area of all peaks, 85% or more of the total area of all peaks, 86% or more of the total area of all peaks, 87% or more of the total area of all peaks, 88% or more of the total area of all peaks, 89% or more of the total area of all peaks, 90% or more of the total area of all peaks, 91% or more of the total area of all peaks, 92% or more of the total area of all peaks, 93% or more of the total area of all peaks, 94% or more of the total area of all peaks, 95% or more of the total area of all peaks, 96% or more of the total area of all peaks, 97% or more of the total area of all peaks, 98% or more of the total area of all peaks, or 99% or more of the total area of all peaks.

In some embodiments, a pharmaceutically acceptable immune adjuvant composition is provided comprising the substantially pure QS-21 xylose-enriched saponin isomer disclosed herein. In some embodiments, a pharmaceutically acceptable immune adjuvant composition is provided comprising a substantially pure QS-21 apiose-enriched saponin disclosed herein. In some embodiments, a pharmaceutically acceptable immune adjuvant composition is provided comprising a substantially pure QS-21 saponin composition disclosed herein. In some embodiments, the disclosure provides a pharmaceutically acceptable immune adjuvant composition comprising a substantially pure QS-21 saponin composition disclosed herein. In some embodiments, the disclosure provides a pharmaceutically acceptable immune adjuvant composition disclosed herein, wherein the composition has an increased adjuvanticity compared to an adjuvant composition comprising a QS-21 apiose saponin isomer and a QS-21 xylose saponin isomer in a ratio of about 65:35. In some embodiments, the disclosure provides a pharmaceutically acceptable immune adjuvant composition disclosed herein, wherein adjuvanticity is measured by an assay comprising induction of IgG antibody production. In some embodiments, the disclosure provides a pharmaceutically acceptable immune adjuvant composition disclosed herein, wherein adjuvanticity is measured by an assay comprising cytokine secretion profiling. In some embodiments, the disclosure provides a pharmaceutically acceptable immune adjuvant composition disclosed herein, wherein adjuvanticity is measured by an assay comprising CD4+ T-cell proliferation. In some embodiments, the disclosure provides a pharmaceutically acceptable immune adjuvant composition disclosed herein, wherein adjuvanticity is measured by an assay comprising expression of CD25 activation marker. In some embodiments, the disclosure provides a pharmaceutically acceptable immune adjuvant composition disclosed herein, wherein the composition has substantially similar or less toxicity compared to an adjuvant composition comprising a QS-21 apiose saponin isomer and a QS-21 xylose saponin isomer in a ratio of about 65:35. In some embodiments, the disclosure provides an immunogenic composition comprising a pharmaceutically acceptable immune adjuvant composition disclosed herein, and an immunogen or antigen, or a polynucleotide encoding the immunogen or antigen.

The disclosure also provides a process for obtaining a substantially pure QS-21 xylose saponin isomer disclosed herein; a substantially pure QS-21 apiose saponin isomer disclosed herein; a substantially pure QS-21 saponin composition disclosed herein; the process comprising purifying a Quillaja saponaria extract by a high-performance liquid chromatography (HPLC) separation process comprising: a reverse phase HPLC separation step, and a normal phase HPLC separation step. In some embodiments of the process, the reverse phase HPLC step comprises use of a phenyl-hexyl stationary phase. In some embodiments of the process, the reverse phase HPLC step comprises use of a sulfobetaine stationary phase. In some embodiments, the process further comprising one or more steps selected from: collecting raw material from pruning trees of the Genus Quillaja; milling the raw material; chipping the raw material; providing an aqueous of the milled and/or chipped raw material; filtering an aqueous extract; concentrating an aqueous extract; a clarification step; one or more filtering steps; and/or one or more concentrating steps.

In other embodiments, a process for obtaining QS-21, wherein the QS-21 will have a xylose-rich ratio ranging from 40-99.9%. Said another way, the ratios can been set forth in percentages, wherein a modified QS-21 compound will have from 40% to 99.9% xylose isomer compared to 60% and 0.1%, respectively, of the apiose isomer. In particular, the modified QS-21 compound has unexpectedly been found to improve adjuvant activity as compared to the standard QS-21.

As either a part of the process above, or separately therefrom, the present invention describes a process for obtaining QS-21 from different sources of the tree, Quillaja Saponaria. Further processes include the use and application of QS-21 and modified QS-21 as a standalone immune adjuvant or in an immune adjuvant system with the same or improved adjuvanticity than the standard QS-21 or for use as a cancer therapeutic, which is only obtained from the bark of the Quillaja tree.

### Example 1: Evaluation of Adjuvanticity of QS-21 with a Standard Composition (65:35-Apiose:Xylose and 75:25-Apiose:Xylose) and Two QS-21 Modified Biomass-Derived Compositions with Isomers Fractions (80:20-Apiose/Xylose) and (3.3:96.7-Apiose/Xylose) in an OVA Murine Mode

The QS-21 saponin used as a standalone adjuvant enhances humoral and cellular immunity against different types of antigens. Additionally, QS-21 used as an adjuvant in licensed vaccines to date is produced solely from the bark of the Quillaja saponaria tree and contains a standard isomeric composition of 65:35 apiose/xylose ratio. The objective of this study was to evaluate and compare the standalone adjuvant activity of two standard QS-21 products derived from Quillaja Saponaria within a natural occurring isomeric composition variation of 75:25 and 65:35 V1:V2. Additionally, the standalone adjuvant activity of two QS-21 products formulated with isomeric composition ratios of 80:20 and 3.3:96.7 V1:V2 derived from biomass were evaluated to compare the differences in the apiose/xylose ratio has on the adjuvanticity of the QS-21 compositions.

C57/BL6 mice (6-8 week-old females, 7 groups, 9 per group) were immunized intramuscularly (Day 0) with 10 μg chicken egg albumin (ovalbumin) in saline with and without the different QS-21 compositions (20 μg mcg). To distinguish the effect of the adjuvants, a control group was added and immunized with the OVA antigen alone to rule out a nonspecific-specific antigen response induced by the adjuvant and lastly a sham saline-only phosphate buffered saline (PBS) (50 μl) group was included to emulate the physical effect of the inoculation on the tissue. At 21 days, a booster immunization was given and the 50 μl blood, popliteal lymph nodes and spleen samples were recovered from all immunized mice and analyzed at 14 days after the booster immunization to enable the formation of immune memory cells. At the end of OVA stimulation, CD4+ T cell proliferation and activation were analyzed by cytometry and the concentration of cytokines secreted by splenocytes was analyzed by CBA (Cytometry Bead Array).

Nomenclature:
QS-21 standard 1: QS-21 (>95% purity) with isomeric composition (65% apiose/35% xylose);
QS-21 standard 2: Standard (>95% purity) with isomeric composition (75% apiose/25% xylose);
QS-21 80:20: QS-21 (>95% purity) obtained from biomass modified in apiose isomer. Isomeric composition (80:20% apiose:xylose)
QS-21 3.3:96.7: QS-21 (>95% purity) obtained from biomass modified in xylose isomer. Isomeric composition (3.3:96.7% apiose:xylose %).

A summary of the results can be found in Table 1.

TABLE 1
Composition of the different immunizations evaluated
in this study with their respective controls.

| Group | | |
|---|---|---|
| V1:V2 ratio | Antigen/ug | QS-21/ug |
| 1. PBS | — | — |
| 2. OVA | 10 | — |
| 3. 65:35 | — | 20 |
| 4. 65:35 | 10 | 20 |
| 5. 75:25 | 10 | 20 |
| 6. 3.3:96.7 | 10 | 20 |
| 7. 80:20 | 10 | 20 |

Further comments:
1 Immunological Analysis Levels of IgG antigen-specific antibodies directed against the antigen the induction of OVA-specific IgG isotype antibody production was measured by ELISA.
2. The T cell proliferation of T cells previously sensitized with the antigen plus the adjuvant and re-stimulated with the same antigen was measured by cytometry.
3. The T cell activation. Evaluated through the measure of surface cell protein expression of activation markers CD25 was measured by cytometry.

### Methods

| Measure/procedure | Protocol/technique |
|---|---|
| Method of Collecting | Blood collection by tail vein bleed |
| Murine Peripheral Blood | |
| Total IgG antibodies | Indirect ELISA. |
| Splenectomy, splenocytes | Isolation of mouse splenic |
| culture and ex vivo re- | T cells for ex vivo T cell receptor |
| stimulation | stimulation assays |
| CD4⁺ T cell proliferation | Lymphocyte proliferation in vitro |
| | with the intracellular fluorescent |
| | dye carboxyfluorescein diacetate |
| | succinimidyl ester. |
| CD4⁺ T cell activation | Cytometry |

### Results

A. Immunization with the Two Standard QS-21 Adjuvants and Both Isomeric Formulations Plus OVA Protein Induce a Higher Production of Specific IgG Antibodies Against OVA than in Absence of Adjuvant.

As shown in FIG. 19, in all cases production of specific IgG antibodies against OVA was greater in the presence of the adjuvants. One-way ANOVA with Bonferroni's multiple comparison test was used for statistical analyses (*p<0.05) for each dilution resulting in a statistical difference between the xylose-enriched QS-21 3.3:97 formulation and the standard 75:25 V1:V2. There were no differences found between the both standards with QS-21 enriched in apiose (80:20 V1:V2). FIG. 19 illustrates antibody responses against the OVA antigen in mice vaccinated with different adjuvants.

1. Immunization with QS-21xyl Adjuvant Plus OVA Protein Induces Greater Proliferation and Activation of CD4⁺ T Cells Compared to the Rest of the Experimental Groups.

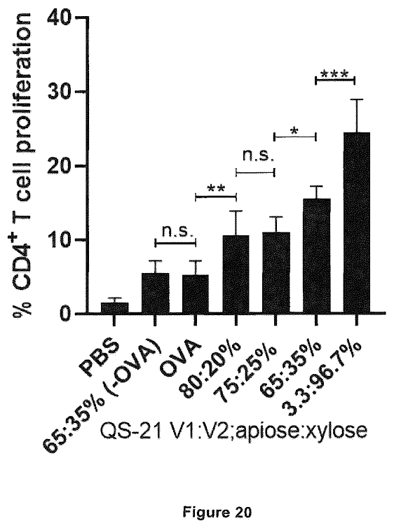

Immunization with the xylose isomer enriched adjuvant plus OVA protein induces greater proliferation and activation of CD4+ T cells compared to the rest of the experimental groups (FIG. 20). The adjuvant effect in the induction of cellular immune response was greatest in the 3.3:96.7 group and statistically different from all the other treatments. There was no difference between the standard (75:25) group and the apiose enriched formulation (80:20).

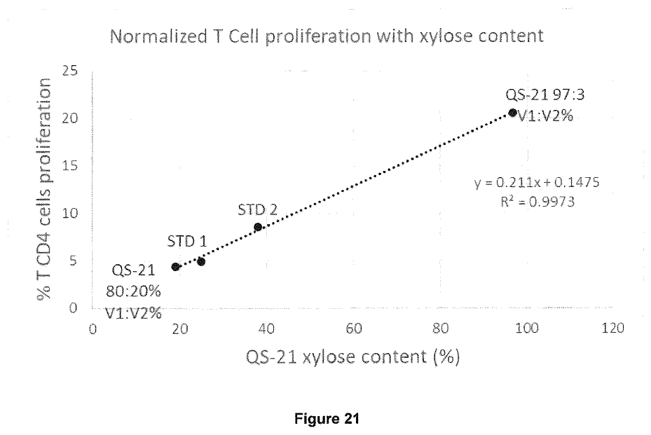

As is depicted in FIG. 20, the group immunized with the xylose enriched formulation presented a greater proliferation of CD4⁺ T cells compared to the rest of the experimental groups. FIG. 21 depicts the correlation between T Cell proliferation (Normalized) with xylose content in QS-21 evaluated products (QS-21 75:25, QS-21 65:35, QS-21 apiose enriched 80:20 and QS-21 xylose enriched 3.3:96.7, V1:V2). FIG. 21 shows a positive linear correlation found (R²=0.9973) between the % of xylose in the tested QS-21 and the % of T CD4 lymphocytes proliferation. Without being bound by theory, this appears to indicate that the QS-21 xylose isomer has more activity in gathering the proliferation of the T CD4 lymphocytes.

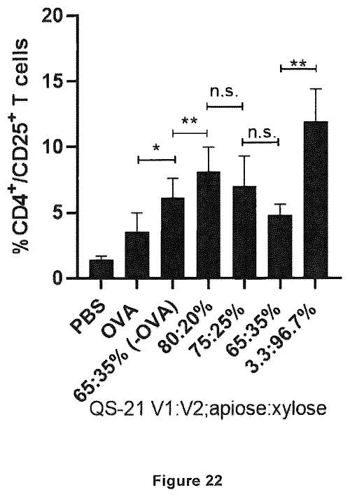

In addition, the group immunized with the xylose enriched formulation presented a higher expression of the CD25 activation compared to the other experimental groups (FIG. 22). The expression of CD25 on T-cells indicate immune activation. The CD25 marker is a component of the IL-2 receptor and play a role in T cell proliferation, activation, as well as the function of effector (Teff) T cells. FIG. 22 depicts the percentage of OVA-specific CD4⁺/CD25⁺ T cells. The group immunized with the xylose-enriched formulation presented a higher expression of the CD25 activation compared to the other experimental groups. The expression of CD25 on T-cells indicate immune activation. The CD25 marker is a component of the IL-2 receptor and play a role in T cell proliferation, activation, as well as the function of effector (Teff) T cells.

One-way ANOVA with Bonferroni's multiple comparison test was used for statistical analyses (*p<0.05) for each group. FIG. 21 shows a strong correlation (R²=0.9973) between the CD4 cells proliferation and the xylose content in the evaluated QS-21 products which can be interpreted as a boost in the immune potency induced at higher xylose isomer content.

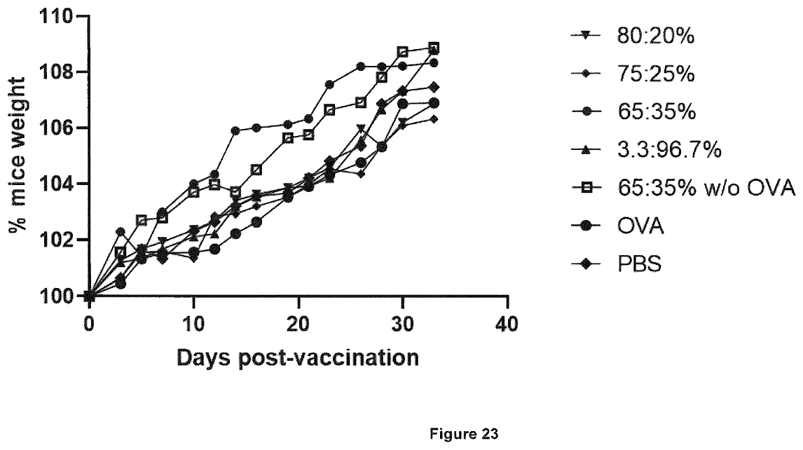

There was no toxicity in any of the treatments tested. As is depicted in FIG. 23, no significant difference was observed in the weight curves of the immunized mice. There was no mortality after any treatment or after any boost nor were behavioral changes observed. The inoculation site did not show any signs of necrosis or exacerbated inflammation. There was no formation of erythema or wound with any of the treatments. FIG. 23 depicts weight curves of mice immunized with the different treatments.

No significant differences were observed between the average weights of the mice immunized with any of the treatments. One-way ANOVA with Bonferroni's multiple comparison test was used for statistical analyses (*p<0.05) for each day.

In conclusion, all QS-21 treatments were potent inducers of anti-OVA IgG production in vaccinated mice. The most potent inducer of CD4⁺ T cells activation was the xylose-enriched group, with almost twice as much CD4⁺ T cell activation compared to the other treatment groups. Regarding the T CD4⁺ activation, all adjuvanted treatment groups showed higher activation levels through the CD25 receptor expression than without adjuvant. The xylose-enriched formulation showed approximately twice as much CD25 receptor expression level than the rest of the groups.

A positive linear correlation was found between the % of xylose in the evaluated QS-21 products and the % of T CD4⁺ lymphocyte proliferation. This would indicate that the QS-21 xylose isomer (V2) has more activity in gathering the proliferation of the T CD4 lymphocytes compared to the QS-21 apiose isomer (V1). These novel results encourage the development of new QS-21 isomeric formulations with an increased QS-21 xylose content directed to give a stronger activity in the generation of this antigen-specific helper T cells and also a greater activation of these cells due the increased of CD25 receptor expression without an increase in toxicity or any inoculation site necrosis or exacerbated inflammation.

### 2: Adjuvanticity and Toxicity Evaluation of Six Different QS-21 (Apiose:Xylose) Isomer Ratio Formulations.

In order to substantiate the adjuvanticity and toxicity results of Example 1, a second OVA mouse study was conducted utilizing six different QS-21 formulations with varying apiose:xylose ratios and a reduced adjuvant dose.

### Materials and Methods

C57/BL6 mice (6-8-week-old females, 8 groups, 9 per group) were immunized intramuscularly (Day 0) with 10 μg chicken egg albumin (ovalbumin) in saline with and without the

| Groups | | |
|---|---|---|
| (V1:V2 ratio) | Antigen/μg | QS-21/μg |
| 99.9:.1% | 10 | 16 |
| 75:25% | 10 | 16 |
| 65:35% | 10 | 16 |
| 51:49% | 10 | 16 |
| 25:75% | 10 | 16 |
| .1:99.9% | 10 | 16 |
| OVA | 10 | — |
| PBS | — | — |

different QS-21 compositions (16 μg total mass). To distinguish the effect of the adjuvants, a control group was added and immunized with the OVA antigen alone to rule out a specific antigen response induced without adjuvant and lastly a sham saline-only phosphate buffered saline (PBS) (50 μl) group was included to emulate the physical effect of the inoculation on the tissue. At 21 days, a booster immunization was given and the 50 μl blood, popliteal lymph nodes and spleen samples were recovered from all immunized mice and analyzed at 14 days after the booster immunization to enable the formation of immune memory cells. At the end of OVA stimulation, CD4+ T cell proliferation and activation were analyzed by cytometry.

More details on the study design are set forth in Table 2.
Table 2. Isomeric ratios of the different QS-21 formulations and dose levels are shown.

A pain, appearance and behavioral assessment guideline (adapted from Morton and Griffiths 1985, Members, Kohn et al. 2007, Carbone 2019, Foley, Kendall et al. 2019) was used to assess animal welfare. This guideline includes the measurement of: 1. body weight variation, 2. appearance, 3. spontaneous behavior, 4. behavior in response to stimuli, 5. appearance of the inoculation site.

### Immunological Read-Outs

1. Isotype IgG ova-specific antibody levels by ELISA. The procedure was the same detailed in Materials & Methods of Example 1.
2. Spleen collection, processing for splenocytes culture and measurement of cell proliferation of specific CD4+ T cells in response to restimulation with the vaccine antigen by CFSE staining. The procedure was identical to the previously detailed in Material & Methods of Example 1.

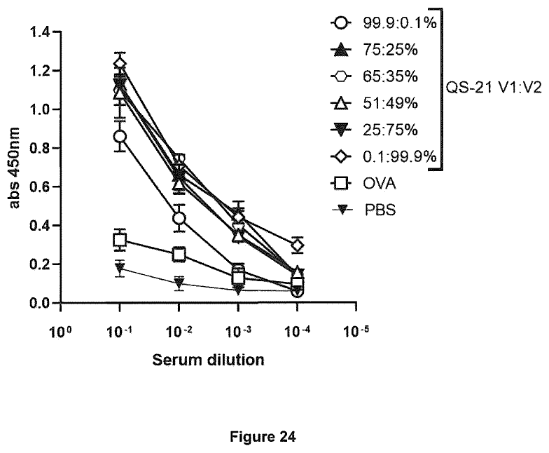

Results: Mice immunized with OVA plus four different isomers ratio formulations of QS-21 induced a significantly high production of antigen-specific antibodies compared to OVA alone. FIG. 24 shows xylose-rich QS-21 formulations produced significantly highly production of antigen-specific antibodies with higher values at higher QS-21 xylose content.

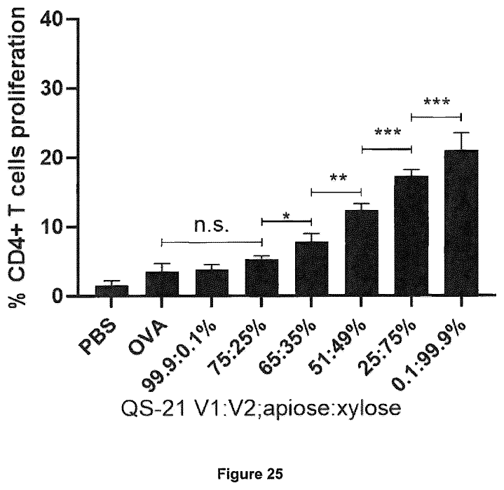

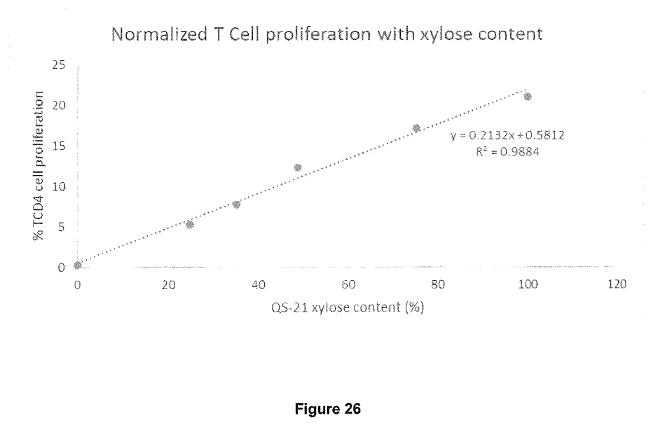

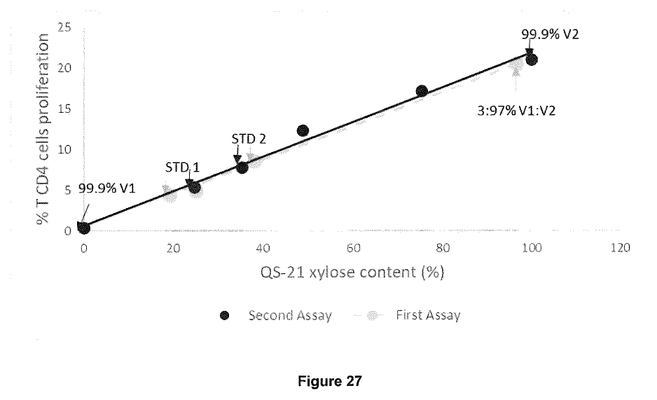

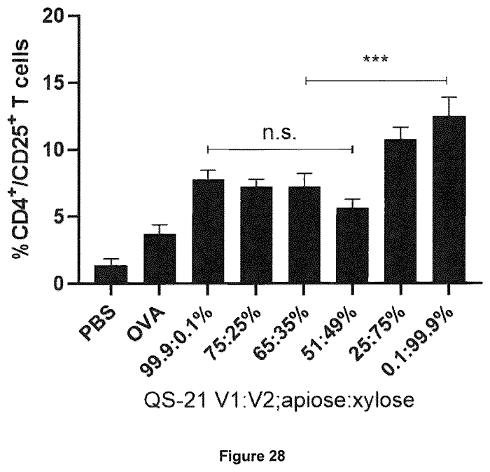

FIG. 25 shows that xylose-rich QS-21 formulations produced a proportional increase in the antigen-specific lymphoproliferative response of CD4+ T cells. FIG. 26 shows a positive linear correlation (R2=0.9884) between the % of xylose in the tested QS-21 isomers formulations and the % of T CD4 lymphocytes proliferation. FIG. 27 shows an overlap of Example 1 and Example 2 between normalized T Cell proliferation and QS-21 xylose content. In addition, FIG. 28 shows the expression of the CD25 activation of T Cells is higher in the xylose-enriched groups. There was no difference between any of the groups regarding body weight variation, appearance, spontaneous behavior, behavior in response to stimuli, or appearance of the inoculation site.

FIG. 24 depicts levels of antigen-specific antibodies against OVA measured by ELISA. One-way ANOVA with Bonferroni's multiple comparison test was used for statistical analyses (*p<0.05). FIG. 25 depicts percentage of proliferation of CD4+ T cells from splenocytes of mice vaccinated with OVA (10 ug) antigen plus QS-21 adjuvant (16 μg) with varying apiose/xylose ratios. One-way ANOVA with Bonferroni's multiple comparison test was used for statistical analyses (*p<0.05). FIG. 26 depicts the correlation between T Cell proliferation (Normalized) with xylose content in six different isomeric QS-21 apiose:xylose proportion formulations. FIG. 27 depicts a comparison between normalized T Cell proliferation and QS-21 xylose content in formulations evaluated in the assays presented in Example 1 and Example 2. FIG. 28 depicts percentage of OVA specific T Cell which express the Activation marker CD25 pulsed in vitro with OVA antigen. One-way ANOVA with Bonferroni's multiple comparison test was used for statistical analyses (*p<0.05).

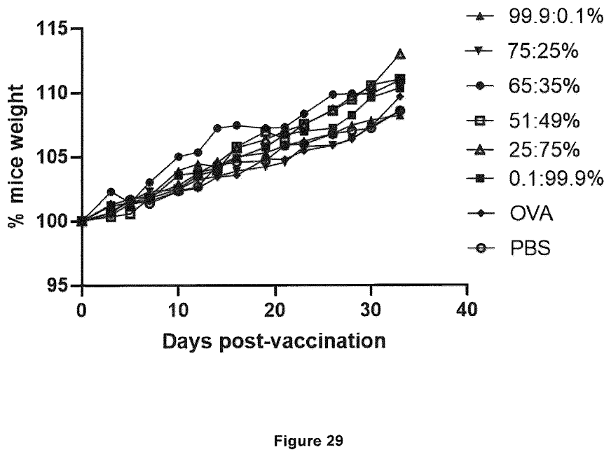

FIG. 29 depicts a weight % curve of each group of animals during the course of the immunization assay for Example 2. As depicted in FIG. 29, no significant difference was observed in the weight curves of the immunized mice. In addition, there was no mortality or behavioral changes observed after any treatment or after any boost. The inoculation site did not show any signs of necrosis or exacerbated inflammation. There was no formation of erythema or a wound with any of the groups treated. This result again suggests that the proportion of QS-21 isomers in the tested formulations does not have a toxic effect and that the tolerability is the same as QS-21 naturally encountered.

In conclusion, the results observed in Example 1 were replicated in Example 2. All QS-21 treatments were potent inducers of anti-OVA IgG production in vaccinated mice and the xylose-enriched groups were the most potent inducers of CD4+ T cells proliferation and activation compared to the other treatment groups. Furthermore, the different isomeric ratio QS-21 formulations did not negatively affect any animal welfare and showed there was no difference in toxicity or tolerability compared to standard QS-21 (65:35), which agrees with the results obtained in Example 1.

Although the present invention has been described in considerable detail with reference to certain embodiments, other embodiments are possible. For example, other antigens other than OVA can be used in the present invention.

### 3: Adjuvanticity Evaluation of Four Different QS-21 Isomer Ratio Formulations Using SARS-COV-2-Derived RBD Antigen.

Study the adjuvant effect of varying QS-21 isomeric formulations using SARS-COV2-derived RBD antigen.

### Materials and Methods

C57/BL6 mice (6-8 week-old females, 8 groups, 9 per group) were immunized intramuscularly (Day 0) with 4 μg RBD SARS-COV-2 antigen in PBS with and without the different QS-21 compositions (16 μg). To distinguish the effect of the adjuvants, a control group was added and immunized with the RBD antigen alone to rule out the induction of immunity induced only by the inoculation of the antigen without the participation of the adjuvant and a sham saline-only phosphate buffered saline (PBS) (50 μl) group was included to emulate the physical effect of the inoculation on the tissue. At 21 days, a booster immunization was given and the 50 μl blood, popliteal lymph nodes and spleen samples were recovered from all immunized mice and analyzed at 14 days after the booster immunization to enable the formation of immune memory cells. At the end of RBD stimulation, CD4⁺ T cell proliferation and activation were analyzed by cytometry.

More details about the treatments assayed in Example 3 are set forth in Table 3.

TABLE 3
Isomeric ratios of the different QS-21
formulations and dose levels are shown.

| Groups | | |
|---|---|---|
| (V1:V2 ratio) | Antigen/μg | QS-21/μg |
| 99.9:.1% | 4 | 16 |
| 75:25% | 4 | 16 |
| 65:35% | 4 | 16 |
| 50:50% | 4 | 16 |
| 30:70% | 4 | 16 |
| .1:00.9% | 4 | 16 |
| RBD | 4 | — |
| PBS | — | — |

### Results: Total IgG Levels Induced Against RBD

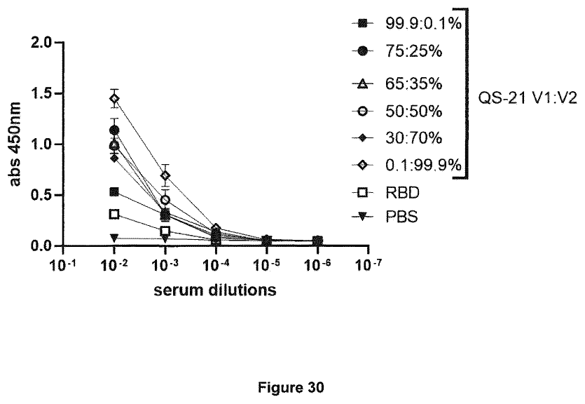

As depicted in the FIG. 30, mice immunized with receptor binding domain (RBD) antigen plus 6 different isomer ratios showed different antibody levels. The highest level of specific RBD antibodies was induced in the 0.1:99.9% group. The 30:70%, 50:50%, 65:35 and 75:25% groups were all lower in antibody production than the 0.1:99.9% group but similar to each other. The 99.9:0.1% group had the lowest level of specific RBD antibodies observed, however all groups had higher levels of antibodies than those induced in the group vaccinated with antigen alone (RBD group).

FIG. 30 depicts the measurement of total IgG RBD-specific antibody levels present in the sera of vaccinated mice of different groups.

### Lymphoproliferative Capacity of CD4⁺ T Cells

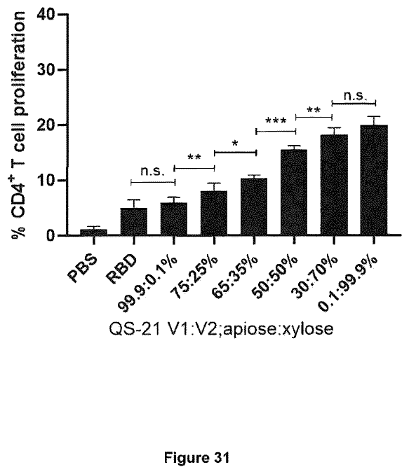

The increase of the xylose isomer of QS-21 used as a standalone adjuvant in RBD immunized mice induces a proportional increase in the antigen-specific lymphoproliferative response of CD4⁺ T cells. Measuring secondary proliferative response in a splenocyte culture of the previously sensitized CD4⁺ T cell population to a new stimulus with the RBD antigen, a positive correlation was observed between lymphoproliferative capacity and the xylose isomer content used as part of the QS-21 adjuvant (FIG. 31). This shows that the xylose isomer content of QS-21 in the vaccine formulation increases the amount of memory CD4⁺ T cells induced upon vaccination and also increases lymphoproliferative capacity in a secondary response. Additionally, no significant differences were observed in this parameter between mice immunized with RBD alone versus mice immunized with a QS-21 adjuvant formulation containing substantially apiose isomer (99.9:0.1% group). FIG. 31 depicts the percentage of proliferation of CD4+ T cells from splenocytes of mice vaccinated with 4 μg of RBD antigen plus QS-21 adjuvant (16 μg) with varying apiose:xylose ratios. One-way ANOVA with Bonferroni's multiple comparison test was used for statistical analyses (*p<0.05).

### Activation of CD4⁺ T Cells

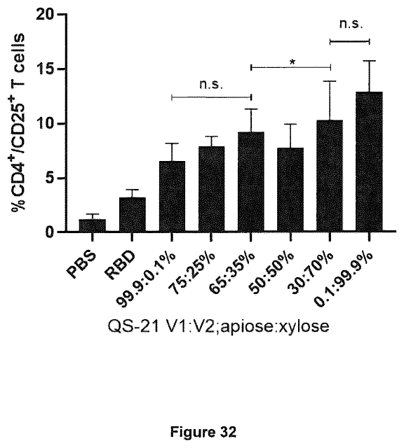

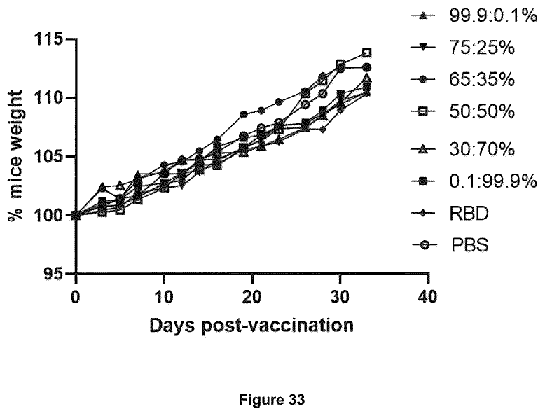

Additionally, it was observed that an increase in the xylose isomer of QS-21 in the vaccine formulation favors the activation state of CD4⁺ T cells in a secondary response. This effect was observed mainly in the groups containing more than 50% of the xylose isomer of QS-21 (FIG. 32). FIG. 32 depicts the percentage of CD4+/CD25+ T cells from splenocytes of mice vaccinated with 4 μg of RBD antigen plus QS-21 adjuvant (16 ug) with varying apiose:xylose ratios. FIG. 33 depicts weight % curve of each group of animals for Example 3. As shown in FIG. 33, no significant weight loss was observed in the groups vaccinated with the isomeric formulations of QS-21 used in Example 3. Also, no toxic effects or significant behavioral changes were observed between the groups.

In conclusion, the immune system behavior observed in Example 3 is similar to Example 2 and all QS-21 treatments were potent inducers of anti-RBD IgG production in vaccinated mice. The 99.9:0.1 apiose-enriched group induced a significantly lower RBD-specific antibody titer than the rest of the treatments. The xylose-enriched group (0.1:99.9) was the most potent inducer of CD4+ T cells proliferation and activation with almost twice as much CD4+ T cell proliferation and with the highest level of activation compared to the other treatment groups. This shows the induction of cell-mediated immunity adjuvant effect of the xylose isomer is antigen independent.

As discovered in Examples 1 and 2, no significant weight loss, toxic effect or behavioral changes was observed in the groups vaccinated with the isomeric formulations of QS-21 used in Example 3.

### 4: Chemical Identification of QS-21 Isomers

This example has the object to demonstrate that the chemical identity of both isolated QS-21 peaks correspond to the QS-21 apiose (V1) and xylose (V2) isomers.

Methods: Three assays were performed in order to determine each peak's identity: 1) HPLC-MS analysis, 2) HPLC with sulfobetaine column DAD analysis to determine the isolated QS-21 isomer composition and 3) HPLC with a C4 column DAD analysis to determine total saponins profile for the two isolated QS-21 peaks.

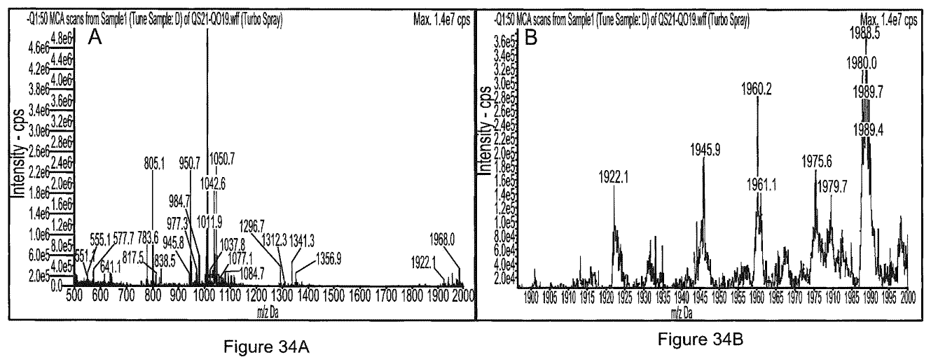

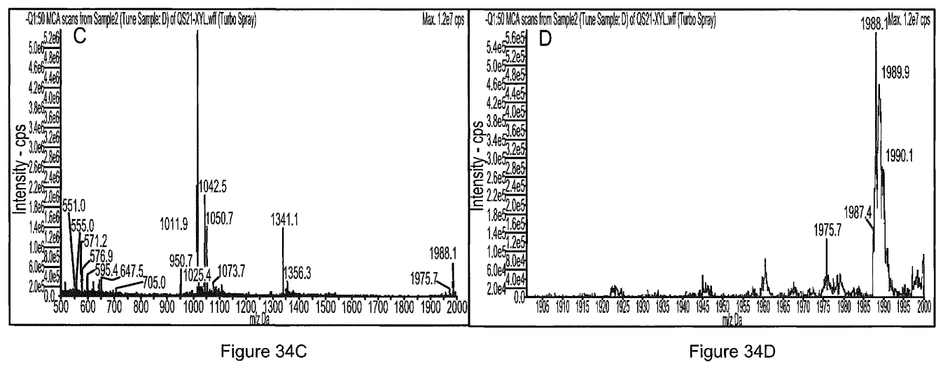

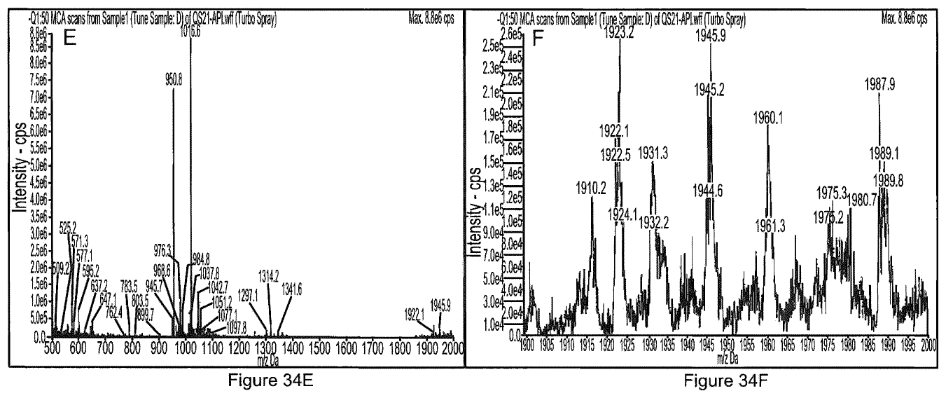

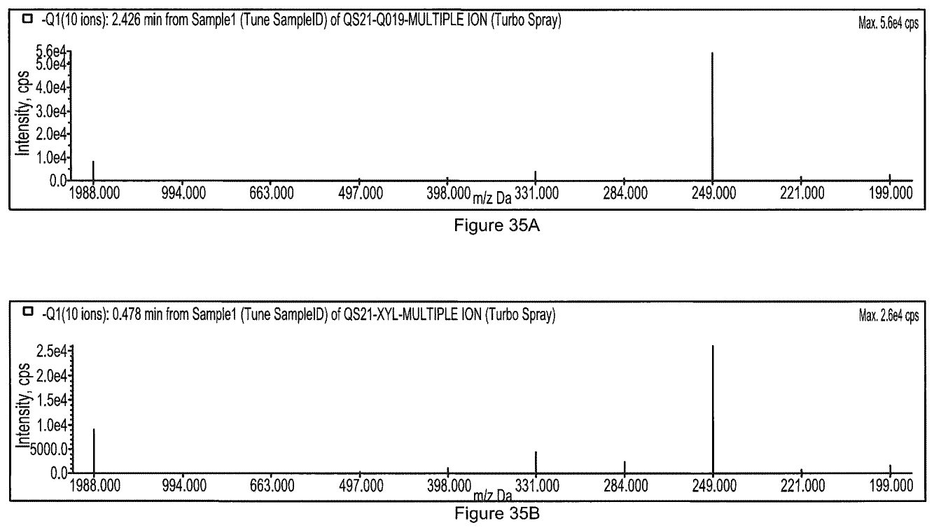

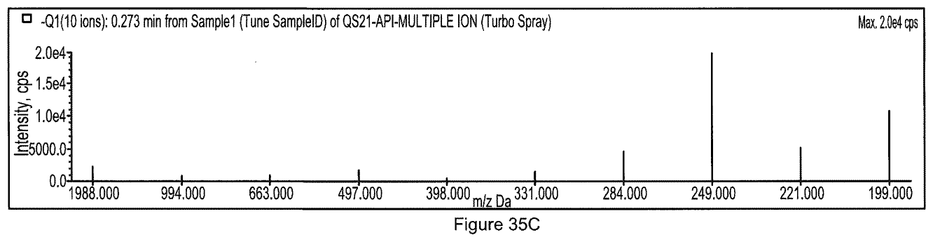

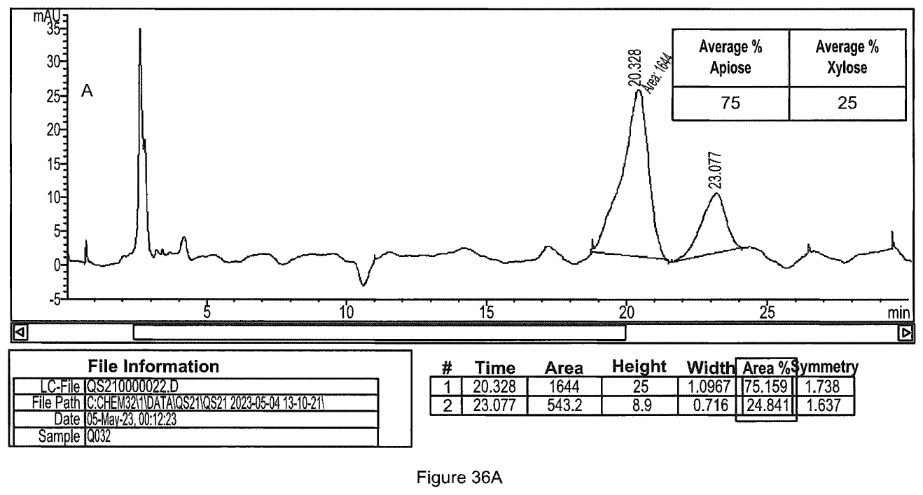

Results: HPLC-MS analysis: Three QS-21 powder samples (standard 75:25, 0:100 xylose enriched, and 100:0 apiose enriched) were dissolved at 1000 ppm in a 35% ACN/65% water plus formic acid at 0.1% solution and infused directly to a mass spectrometer AB Sciex Triple Quad 4500. Two kinds of MS analyses were carried out on each sample. 1) Total Ion Counter (TIC) as shown in FIGS. 34 and 2) Multiple Ions Analysis (MI) depicted in FIG. 35. In FIG. 34, the molecular ion of 1988 and the fragmentation pattern in the three samples was the same, however the intensity of each was different. The differences in intensity can be related to different geometries adopted by each QS-21 isomer. In FIG. 35, the multi-ion analysis also shows similar ionization patterns in the three analyzed samples, where the molecular ion [M-H]-shows a mass of 1988 (with a z value=1) with a major ionization of m/z=249, with a z value=8). This demonstrates that all of the samples have the same molecular weight (1988 Da for [M-H]), which correspond to the QS-21 isomers.

FIG. 34 depicts TICs of QS-21 obtained from standard QS-21 (75:25 V1:V2%) (FIG. 34A), QS-21 xylose-enriched (FIG. 34C) and QS-21 apiose enriched (FIG. 34E). Signal zoomed between 1900-2000 Da for Standard QS-21 (FIG. 34B), QS-21 xylose (FIG. 34D) and QS-21 apiose (FIG. 34F). QS-21 xylose and QS-21 apiose with 100% isomeric purity respectively. FIG. 35 depicts TICs of negative Multiple Ion Analysis (MI) of standard QS-21 (75:25%) of V1:V2 isomer (FIG. 35A), QS-21 xylose enriched (FIG. 35B) and QS-21 apiose enriched (FIG. 35C), with 100% isomeric purity respectively. QS-21 (1988 Da [M−H]−) shows the same molecular ion pattern in the three products (m/z=1988/1; 249/8), noticeable is that m/z majoritarian corresponds to 249 Da, with denotes a z=8 in this methodology for all tested pure QS-21 products. Standard QS-21 (75:25%) of V1:V2 isomer.

HPLC on Sulfobetaine column DAD analysis: The standard QS-21 (75:25) together with the two pure isomers product, was subject to the sulfobetaine analytical methodology for quantifying the isomers using the same method previously disclosed at analytical scale that allows the QS-21 V1 and V2 isomer separation as follows:
Column: Sulfobetaine 250×4.6 mm, 10 μm, 100 Å.
Flow: 1 mL min⁻¹.
Mobile phase: Isocratic 84% ACN/16% water (5 mM ammonium acetate). wavelength detection: 210 nm.

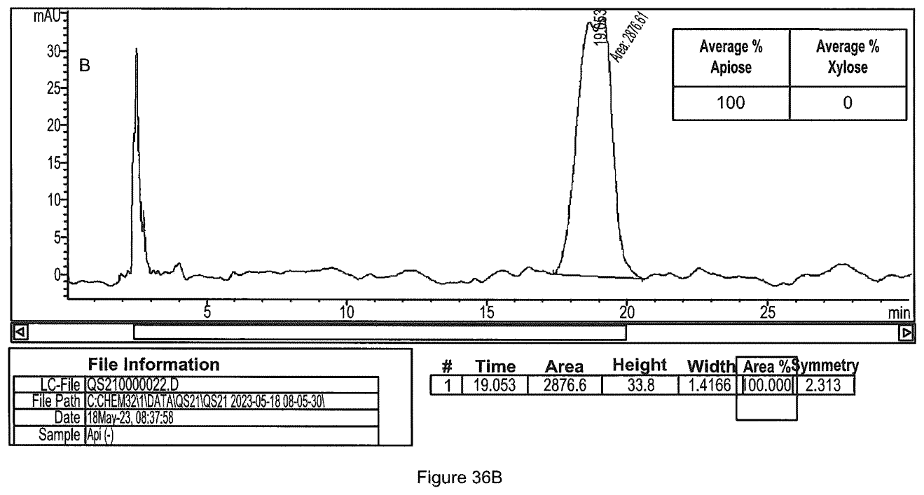

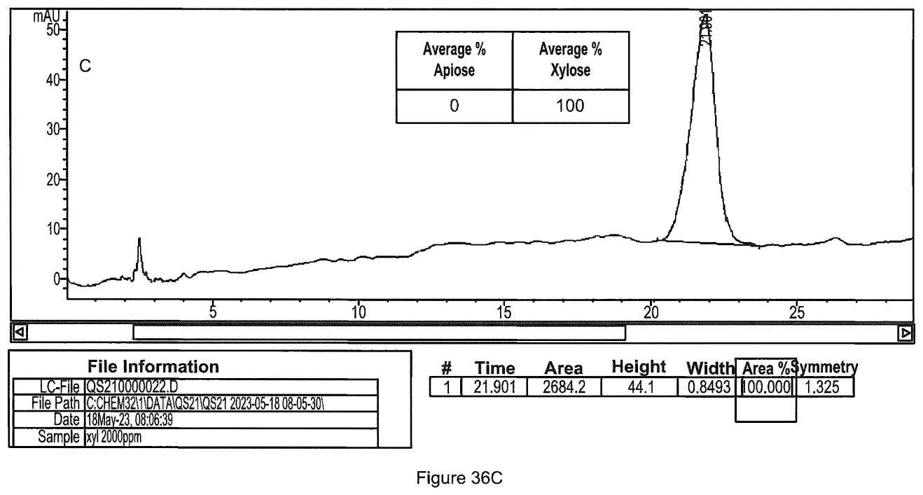

As depicted in FIG. 36, the standard QS-21 (FIG. 36A) is a mix of 75:25% of V1:V2 isomers while the QS-21 apiose and QS-21 xylose shows a 100% isomeric purity with IT of each peak corresponding to 20.3 minutes for apiose and 23.0 minutes for xylose QS-21 isomers respectively (FIGS. 36B and 36C). FIGS. 36A, 36B and 36C depicts HPLC analysis using the sulfobetaine analytical methodology for isomer determination of standard QS-21 (75:25%) of V1:V2 isomer (A), QS-21 apiose (B) and QS-21 xylose (C), with 100% isomeric purity respectively.

HPLC with a C4 column DAD analysis: A HPLC C4 column DAD was used on all three samples (standard 75:25, apiose-only and xylose-only QS-21) to measure total saponin content with the standard quality control methodology, adapted from San Martin & Briones,² as follows: Column: C4 250×4.6 mm, 5 μm, 300 Å.
Flow: 1 mL min⁻¹.

### Solvents:

A: Water+0.15% V/V TFA
B: Acetonitrile+0.15% V/V TFA

### Gradient:

Wavelength detection: 210 nm.

| Time (min) | % A | % B |
|---|---|---|
| 0 | 60 | 40 |
| 12 | 50 | 50 |
| 12.1 | 60 | 40 |
| 16.1 | 60 | 40 |

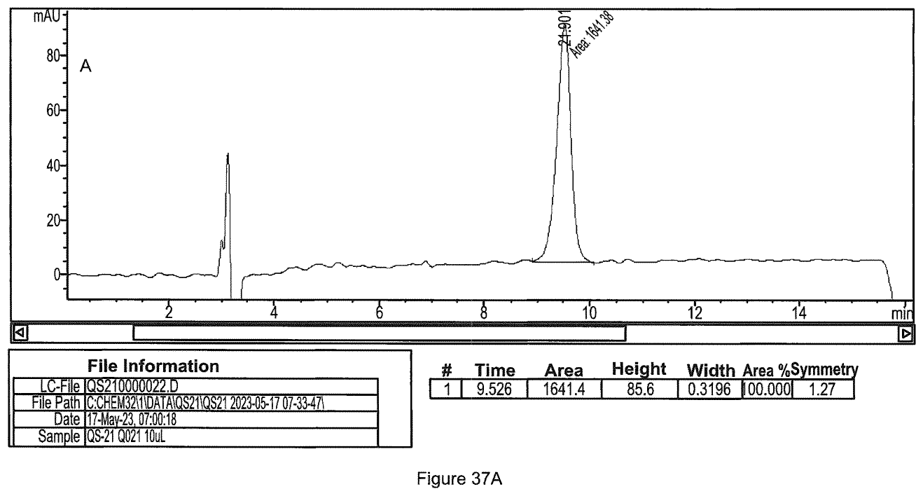

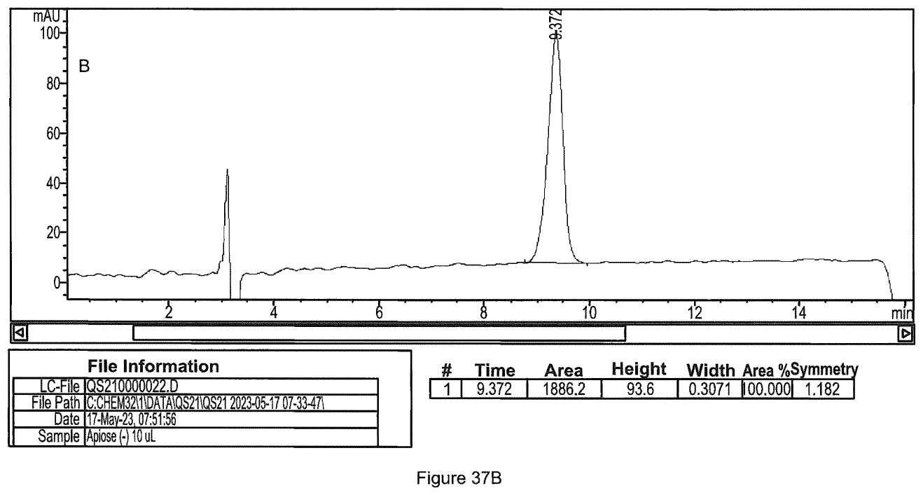

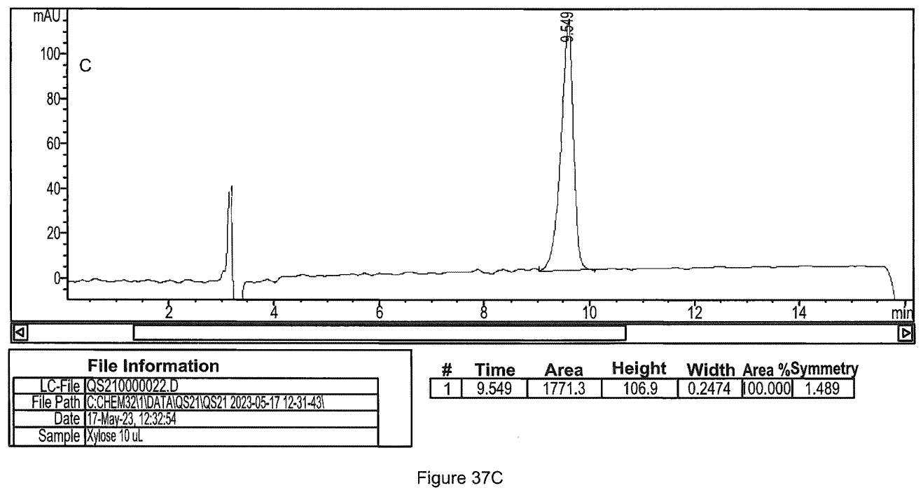

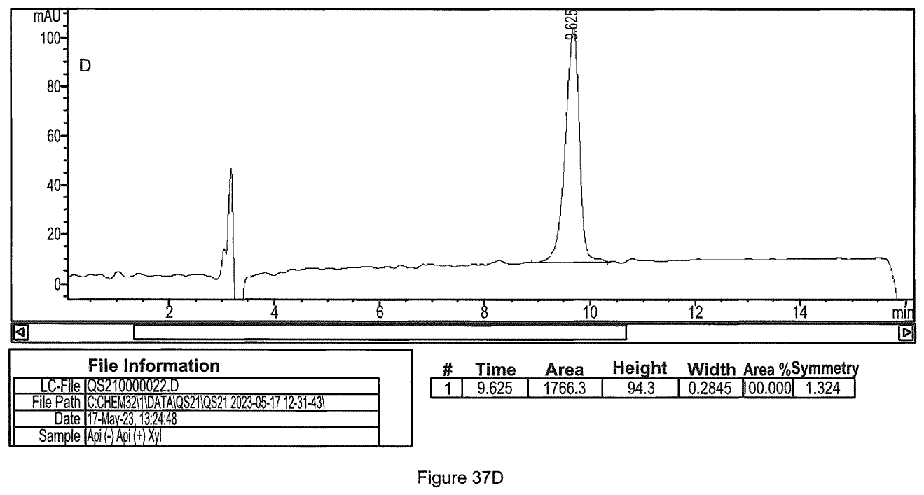

The results show the same peak shape profile and rT (FIGS. 38A,B & C) for all three samples. Even a combination of the three samples at the same concentration (QS-21 standard and both QS-21 isomers), shows a single peak with the exact same rT and shape prolife than standard QS-21, as depicted in FIG. 37D. All three samples show 100% QS-21 saponin purity and demonstrate that each sample has identical retention times. FIG. 37A-D depict an analytical method for total saponin purity by HPLC using a C4 column. A 20 μL of solution of 2000 ppm for each sample was injected accordingly: Standard QS-21 (75:25), QS-21-apiose-enriched (B), QS-21-xylose-enriched (C) Isomers and a Combination 1:1:1 in volume of each product (D).

In conclusion, based on the results obtained from the HPLC-MS and both HPLC-DAD methods, it can be concluded that the compounds isolated are the V1 and V2 isomers of QS-21 respectively and all compounds show over 95% QS-21 saponin purity.

1. A QS-21 saponin composition modified by its isomeric ratio of QS-21 apiose saponin isomer and QS-21 xylose saponin isomer within the composition to provide a modified QS-21 saponin composition comprising: a QS-21 apiose (V1) saponin isomer and a QS-21 xylose (V2) saponin isomer in a ratio ranging between 60:40 (V1:V2) and 0.1:99.9 (V1:V2), whereas a standard QS-21 saponin composition comprises a QS-21 apiose (V1) saponin isomer and a QS-21 xylose (V2) saponin isomer in a ratio ranging between 65:35 (V1:V2) and 75:25 (V1:V2).

2. The QS-21 saponin composition, as claimed in claim 1, purified from a crude Quillaja saponaria extract to provide a pure QS-21 saponin composition comprising a QS-21 apiose saponin isomer and a QS-21 xylose saponin isomer in a ratio of (A parts apiose isomer):(X parts xylose isomer), wherein: 0.1<A<65 and 35<X<99.9, wherein A+X=100; and wherein the pure QS-21 saponin composition is characterized by one or more predominant peaks comprising at least 90%, preferably at least 90% to 95, and more preferably at least 95%, of the total area of all peaks of a chromatogram, excluding the solvent peak, when analyzed on a hydrophilic interaction liquid chromatography (HILIC) using sulfobetaine 250×4.6 mm, 10 μm, 100 Å under Isocratic conditions of a mixture comprising 84% ACN/16% water (5 mM ammonium acetate) at 1 mL min-1 flow rate and 210 nm of wavelength detection.

3. The QS-21 saponin composition, as claimed in claim 1, wherein the ratio of apiose (V1) isomers to xylose (V2) isomers is between 50:50 and 1:99, preferably between 40:60 and 5:95, more preferably between 30:70 and 10:90, and more preferably is between 25:75 and 15:85.

4. The QS-21 saponin composition, as claimed in claim 1, wherein the saponin is taken from the bark and at least one other part of a Quillaja plant.

5. An immunogenic composition comprising:
an adjuvant composition comprising the QS-21 saponin composition as claimed in claim 1, and
an immunogen or antigen, or a polynucleotide encoding the immunogen or antigen.

6. A pharmaceutically acceptable immune adjuvant composition comprising the QS-21 saponin composition as claimed in claim 1.

7. The pharmaceutically acceptable immune adjuvant composition as claimed in claim 6, wherein adjuvanticity is measured by an assay selected from the group consisting of an assay comprising induction of IgG antibody production, an assay comprising CD4+ T-cell proliferation, and an assay comprising expression of a CD25 activation marker.

8. The pharmaceutically acceptable immune adjuvant composition as claimed in claim 6, wherein the adjuvant composition has an increased adjuvanticity and has substantially similar or less toxicity compared to an adjuvant composition comprising a QS-21 apiose saponin isomer and a QS-21 xylose saponin isomer in a ratio of between 65:35 and 75:25.

9. A cancer therapeutic comprising the QS-21 saponin composition as claimed in claim 1.

10. A process for obtaining the QS-21 saponin composition of claim 1, the process comprising purifying a Quillaja saponaria extract by a high-performance liquid chromatography (HPLC) separation process comprising:
a reverse phase HPLC separation step.

11. The process as claimed in claim 10, wherein the reverse phase HPLC step comprises one or more of the following: (1) use of a phenyl-hexyl stationary phase, and (2) use of a sulfobetaine stationary phase.

12. The process as claimed in claims 10 to 11, further comprising one or more steps selected from:
collecting raw material from pruning trees of the Genus Quillaja;
milling the raw material;
chipping the raw material;
providing an aqueous mixture of the milled and/or chipped raw material;
filtering the aqueous mixture to obtain an aqueous extract;
concentrating the aqueous extract;
clarifying the aqueous extract by eliminating solids and purify the saponins from the extract;
further filtering the clarified extract to produce a crude extract; and
concentrating the crude extract to obtain a saponin extract.

13. The process as claimed in claim 12, wherein the step of further filtering includes filtering through a press filter to produce the crude extract and treating the crude extract by nanofiltration techniques to increase the saponin content up to 75 to 99% on a dry basis (ODB).

14. The process as claimed in claim 12, wherein the step of concentrating includes concentrating the crude extract at vacuum, obtaining the saponin extract in an ultra pure form, and treating the ultra-pure saponin extract with a High-Performance Liquid Chromatography (HPLC) separation with orthogonal two sequential steps to obtaining a QS-21 pure fraction.

15. A method of using the QS-21 saponin composition as claimed in claim 1, as one of a standalone immune adjuvant, a composition in an immune adjuvant system, and as a cancer therapeutic.
